(19) United States
(12) Patent Application Publication
(10) Pub. No.: US 2009/0175903 A1
(43) Pub. Date: Jul. 9, 2009

(54) MEASLES SUBUNIT VACCINE

(75) Inventors: Brian J. Ward, Montreal (CA); David S. Burt, Dollard des Ormeaux (CA); Sophie Chabot, St-Damien-de-Buckland (CA)

Correspondence Address: SEED INTELLECTUAL PROPERTY LAW GROUP PLLC, 701 FIFTH AVE, SUITE 5400, SEATTLE, WA, 98104, US

(73) Assignees: ID BIOMEDICAL CORPORATION OF QUEBEC, Laval (CA); MCGILL UNIVERSITY, Montreal (CA)

(21) Appl. No.: 12/182,047

(22) Filed: Jul. 29, 2008

Related U.S. Application Data
(63) Continuation of application No. 10/941,634, filed on Sep. 15, 2004, now abandoned.
(60) Provisional application No. 60/503,114, filed on Sep. 15, 2003.

(51) Int. Cl. A61K 39/295; A61K 39/165; A61P 37/04

(52) U.S. Cl. 424/201.1; 424/212.1; 424/202.1

(57) ABSTRACT

Compositions and methods for making and using therapeutic formulations of measles virus antigens with a Proteosome-based adjuvant are provided. The measles virus antigens may be derived from a variety of sources, such as from recombinant production or from a split antigen preparation. The measles vaccine formulations may be used, for example, in methods for treating or preventing a measles virus infection and eliciting a protective immune response.

### CROSS-REFERENCE TO RELATED APPLICATIONS

This application is a continuation application of U.S. application Ser. No. 10/941,634, filed Sep. 15, 2004, which claims the benefit of U.S. Provisional Patent Application No. 60/503,114 filed Sep. 15, 2003, which applications are incorporated herein by reference in their entirety.

### BACKGROUND OF THE INVENTION

1. Field of the Invention

The present invention relates generally to vaccines and the treatment or prevention of infectious disease and, more specifically, to compositions comprising a Proteosome adjuvant or a Proteosome:liposaccharide adjuvant formulated with measles virus antigens, and therapeutic uses thereof.

2. Description of the Related Art

Measles is a highly communicable disease that infects an estimated 40 million people annually, causing over 900,000 deaths per year (WHO/UNICEF, Joint WHO/UNICEF statement on Vitamin A for Measles, Weekly Epidemiology Record 19:133, 1987). In 2001, the World Health Organization (WHO) and UNICEF announced a program to reduce measles mortality by at least 50% by 2005 through targeted vaccination campaigns in developing countries. This is to be achieved by ensuring greater than 80% coverage in over 80% of the world (Id.; Orenstein et al., Am. J. Public Health 90:1521; 2000). Although the live-attenuated vaccines in current use are effective, they have serious limitations. In particular, they often fail to protect children younger than 9 months of age due to the presence of neutralizing maternal antibodies (Albrecht et al., J Pediatr. 91:715 (1977); Markowitz et al., N. Engl. J. Med. 322:580 (1990)). Between 30% and 50% of measles virus (MV) associated deaths occur during this vulnerable period. Maternal anti-measles antibody titers vary widely, and passively acquired antibodies normally have a half-life of three to four weeks. As a result, infants become susceptible to measles at almost any time between birth and one year in age (Crowe, Clin. Infect. Dis. 33:1720, 2001).

Several attempts have been made to bypass the interference of maternal antibodies using currently available live attenuated vaccines. A high titer, live (infectious) measles virus vaccination, which has up to 10^{6.3} plaque forming units (PFU) of vaccine strain virus as compared to 10^{3.4} PFU for standard vaccination, was tried (Markowitz et al.) and aerosol administration was tried (Bennett et al., Bull. World Health Organ. 80:806, 2002). The former approach could successfully protect children as young as 3 months of age, but it was associated with a poorly understood increase in childhood mortality. Such mortality may be associated with the administration of vaccines containing live or attenuated measles virus. Early results with aerosol administration of live vaccine MV strain were also promising. However, the doses administered needed to be quite high and the delivery systems are very cumbersome. While unacceptable, these attempts demonstrated that a 2-3 month old infant has an intrinsic ability to respond to MV antigens, and that mucosal immunization might be less susceptible to the interference of maternal antibodies.

The ability of antigens to induce protective immune responses in a host can be enhanced by combining the antigen with an immunostimulant and/or adjuvant. Alum-based adjuvants are almost exclusively used for licensed injectable human vaccines. However, while alum enhances certain types of serum antibody responses (Type 2), it is poor at enhancing other types of antibody responses (Type 1) and is a poor activator of cellular immune responses that are important for protection against, for example, intracellular pathogens.

Hence, a need exists for identifying and developing compositions therapeutically effective against measles infections, particularly those compositions that can function as a vaccine and elicit protective immunity. Furthermore, a need exists for vaccine formulations, particularly subunit vaccine formulations, which include potent adjuvants that are safe in humans and capable of enhancing the induction of protective systemic and mucosal humoral and cellular immune responses. The present invention meets such needs, and further provides other related advantages.

### BRIEF SUMMARY OF THE INVENTION

The present invention provides Proteosome formulated measles vaccine compositions, and therapeutic uses thereof. These vaccines are straightforward to produce and are capable of eliciting a protective immune response for treating or preventing a measles infection. Measles antigens may comprise one or more recombinantly or synthetically produced measles polypeptides, or can comprise one or more measles polypeptides isolated from measles viral particles or infected host cells. Measles antigens comprise at least one measles virus polypeptide, such as the measles virus H protein or F protein, or may comprise two or more measles virus antigens, capable of eliciting a neutralizing antibody response or cell mediated immunity. Proteosome formulated adjuvants may comprise outer membrane proteins obtained from Gram-negative bacteria (projuvant) or a combination of outer membrane proteins and liposaccharides (OMP-LPS).

In one aspect, the present invention provides an immunogenic composition, comprising an adjuvant and one or more measles virus antigens, wherein the adjuvant comprises a Proteosome and liposaccharide, and at least one of the measles antigens is an H protein. In certain embodiments, the immunogenic composition comprises at least two measles virus antigens comprising an F protein and an H protein. In other embodiments, the one or more measles virus antigens are recombinant measles antigens or a measles split antigen. In related embodiments, the measles split antigen is from a Moraten, Shwarz, Zagreb, or Edmonston strain of measles virus. In still other embodiments, the liposaccharide final content by weight as a percentage of Proteosome protein of the immunogenic composition ranges from about 10% to 500%. In yet another embodiment, the Proteosomes and liposaccharide are obtained from the same bacteria or are from different bacteria. In other embodiments, the Proteosomes are from Neisseria species. In still other embodiments, the liposaccharide is from Shigella, Plesiomonas, Escherichia, or Salmonella species. In certain embodiments, the immunogenic composition of the present invention further comprises one or more additional microbial antigens, such as a viral antigen, bacterial antigen, parasitic antigen, or a combination thereof. In certain embodiments, any of the aforementioned immunogenic compositions further comprises a pharmaceutically acceptable carrier, excipient, or diluent.

In another embodiment, the present invention provides an immunogenic composition comprising an adjuvant and one or more measles virus antigens, wherein the adjuvant comprises a Proteosome and at least one measles antigen is H protein. In certain embodiments, the composition comprises at least two measles virus antigens comprising an F protein and an H protein. In other embodiments, the one or more measles virus antigen is a recombinant measles antigen or a measles split antigen. In related embodiments, the measles split antigen is from a Moraten, Shwarz, Zagreb, or Edmonston strain. In other embodiments, the Proteosome is from Neisseria meningitidis. In still other embodiments, the ratio of Proteosomes to measles virus antigen is at least 2:1, 3:1, or 4:1. In certain embodiments, the immunogenic composition of the present invention further comprises one or more additional microbial antigens, such as a viral antigen, bacterial antigen, parasitic antigen, or a combination thereof. In other embodiments, any of the aforementioned immunogenic compositions further comprise a pharmaceutically acceptable carrier, excipient, or diluent.

In still another embodiment, the invention provides a method of treating or preventing a measles infection, comprising administering to a subject in need thereof any of the aforementioned immunogenic compositions. In a related aspect, the present invention pertains to a method of eliciting an immune response, comprising administering to a subject in need thereof any of the aforementioned immunogenic compositions. In certain embodiments, the immune response comprises a mucosal immune response. In other embodiments, the immune response comprises a cell-mediated response. In certain embodiments, the aforementioned immunogenic compositions may be administered by a route selected from mucosal, enteral, parenteral, transdermal, transmucosal, intranasal, or inhalation.

In one embodiment, the invention provides a method for eliciting an immune response comprising administering to a subject in need thereof a recombinant expression vector comprising at least one promoter operatively linked to a polynucleotide encoding at least one measles virus antigen, followed by administering at least once the composition of any one of the aforementioned immunogenic compositions. In a certain embodiment, the at least one measles virus antigen is H protein; in another embodiment, the polynucleotide encodes at least two measles virus antigens, which are H protein and F protein. In another embodiment, the method comprises administering the composition intranasally. In certain embodiments the immune response is a systemic humoral response; a mucosal immune response; wherein the mucosal response comprises production of a IgA immunoglobulin; and/or a cell mediated immune response.

In one embodiments, a method is provided for treating or preventing a measles infection, comprising administering a recombinant expression vector comprising at least one promoter operatively linked to a polynucleotide encoding at least one measles virus antigen, followed by administering at least once any one of the aforementioned immunogenic compositions. In a certain embodiment, the at least one measles virus antigen is H protein; in another embodiment, the polynucleotide encodes at least two measles virus antigens, which are H protein and F protein. In another embodiment, the method comprises administering the composition intranasally. In certain embodiments the immune response is a mucosal immune response and in another embodiment the immune response is a cell-mediated response.

In another embodiment, the immunogenic compositions comprising an adjuvant and one or more measles virus antigens described herein may be used for the manufacture of a medicament for treating or preventing a measles infection in a subject. In another embodiment, such immunogenic compositions may be used for the manufacture of a medicament for eliciting an immune response. In certain embodiments the immune response is a systemic humoral response; a mucosal response; wherein the mucosal response comprises production of a IgA immunoglobulin; and/or a cell mediated immune response.

These and other aspects of the present invention will become evident upon reference to the following detailed description and attached drawings. In addition, various references to published documents are set forth herein which describe in more detail certain aspects of this invention, and are therefore incorporated by reference in their entireties.

### BRIEF DESCRIPTION OF THE DRAWINGS

FIGS. 1A and B show two embodiments for the manufacture of Proteosome bulk material (Flow Chart 1A and Flow Chart 1B, respectively).

FIG. 2 shows a scheme for the manufacture of Shigella flexneri 2a LPS (Flow Chart 2).

FIG. 3 shows a scheme for the manufacture of IVX-908 Proteosome-LPS adjuvant (Flow Chart 3).

FIGS. 4A and 4B show that measles virus F protein and H protein are detectable in the measles virus split antigen preparation in an immunoblot analysis. An immunoblot of the measles virus split antigen preparation was probed with an antibody that specifically binds to H protein (FIG. 4A, lane indicated by “H”) and with an antibody that specifically binds to F protein (FIG. 4A, lanes indicated by “F”). The anti-F protein antibody was also used to probe an immunoblot of a Vero cell extract. A Coomassie® blue stained gel of the measles virus split antigen preparation is presented in the far right lane of FIG. 4A.

FIG. 4B represents a quantitative densitometric analysis of the Coomassie® blue stained gel.

FIG. 5 shows analysis of MV antigen preparation, with and without projuvant or OMP-LPS, by SDS-PAGE and electron microscopy. In FIG. 5A, the left panel is a Coomassie® blue stained SDS-PAGE gel of the samples listed below; the middle panel represents an immunoblot probed with an anti-H protein monoclonal antibody; the right panel represents an immunoblot probed with an anti-F protein monoclonal antibody. Lane 1: MV; lane 2: soluble fraction of MV; lane 3: insoluble fraction of MV; lane 4: MV+OMP; lane 5: soluble fraction of MV+OMP; lane 6: insoluble fraction of MV+OMP; lane 7: OMP alone.

FIG. 5B illustrates the presence of H protein in the Proteosome:MV preparation (Pro-MV) and in the OMP-LPS-MV preparation.

FIGS. 6A-6C show graphic representations of levels of serum IgG and mucosal IgA in animals that received MV split antigen vaccines. FIG. 6A represents immunoglobulin levels in mice administered Proteosome:MV intranasally (IN). FIG. 6B represents immunoglobulin levels in mice administered Proteosome:MV intramuscularly (IM). FIG. 6C represents immunoglobulin levels in mice administered OMP-LPS-MV intranasally (IN).

FIG. 7 shows graphic representations of plaque reduction neutralization (PRN) activity of antibodies in sera and mucosal antibodies in nasal and lung washes of animals immunized intranasally with the Proteosome:MV (Pro-MV IN) (top panel); intramuscularly with Proteosome:MV (Pro-MV IM) (middle panel); and intranasally (IN) with OMP-LPS-MV (bottom panel).

FIG. 8 shows graphic representations of levels of specific IgG isotypes as an indicator for the type of T_{H} response. Animals were immunized intranasally with the Proteosome:MV (Pro-MV IN) (top panel); intramuscularly with Proteosome:MV (Pro-MV IM) (middle panel); and intranasally (IN) with OMP-LPS-MV (bottom panel).

FIG. 9 presents immunoblots of MV antigen probed with a monoclonal antibody specific for H protein (first lane); a monoclonal antibody specific for F protein (second lane); a monoclonal antibody specific for M protein. Serum samples with high neutralizing activity in a PRN assay were applied to immunoblots of MV proteins (fourth lane) and Vero proteins (fifth lane), and serum samples with low neutralizing antibody or with low neutralizing activity were applied to immunoblots of MV proteins (sixth lane) and Vero proteins (seventh lane).

FIG. 10 presents immunoblot analysis and electron microscopy analysis of a measles virus split antigen preparation. FIG. 10A shows an immunoblot in which a measles virus split antigen preparation was probed with an anti-H protein monoclonal antibody (lane “H”) or an anti-F protein monoclonal antibody (lane: “F”). The measles virus antigen preparation was also stained with Coomassie® Blue (lane “Coomassie”). FIG. 10B presents a densitometry analysis of protein bands detected by SDS-PAGE. FIG. 10C illustrates an electron microscopy analysis of IVX908 alone (left panel) and IVX908 combined with the measles virus antigen preparation and detected with an anti-H protein monoclonal antibody.

FIG. 11 illustrates quantification by ELISA of IgG in sera of mice immunized with varying doses of IVX908-MV. FIG. 11A: ng/ml of MV-specific IgG in sera obtained from animals at 14, 28, and 38 days after immunization. FIG. 11B, left panel: detection of IgG1 and IgG₂ₐ in sera; FIG. 11B, right panel: ratio of IgG1:IgG₂ₐ in mice.

FIG. 12 presents ELISA data illustrating the level of IgA in nasal and lung washes obtained from animals 10 days after the last immunization with IVX908-MV, which was at day 24 for animals receiving two doses (FIG. 12A) or at day 38 for animals receiving three doses (FIG. 12B). Statistical significance denoted by * indicates p<0.05 by one way analysis of variance (ANOVA) analysis and Bonferroni multiple comparisons test.

FIG. 13 presents a graphic representation of plaque reduction neutralization activity of serum samples obtained from animals after receiving two doses (FIG. 13A) and three doses (FIG. 13B) of IVX908-MV. Statistical significance denoted by * indicates p<0.05 by one way analysis of variance (ANOVA) analysis and Bonferroni multiple comparisons test.

FIG. 14 illustrates an immunoblot analysis for determining the presence of antibodies that specifically bind to measles virus antigens in sera collected from IVX908-MV-immunized mice. First lane: split MV antigen preparation blotted with an anti-H protein monoclonal antibody; second lane: split MV antigen preparation blotted with an anti-F protein monoclonal antibody; third lane: split MV antigen preparation blotted with mouse sera; fourth lane: Vero cell preparation blotted with mouse sera; fifth lane: Proteosome blotted with mouse sera. Molecular weights of the proteins detected are as follows: measles virus H protein (80 kDa); measles virus F₀ protein (50-60 kDa); measles virus F₁ protein (41 kDa); N. meningitidis OMP Por A (45 kDa); and N. meningitidis OMP Por B (33 kDa).

FIG. 15 illustrates interferon gamma (IFNγ) production in splenocytes isolated from mice that received 2 doses (FIG. 15A) and 3 doses of IVX908-MV (FIG. 15B) and then stimulated with MV split antigen. Statistical significance denoted by * indicates p<0.05 according to T-test one tail of unequal variances.

### DETAILED DESCRIPTION OF THE INVENTION

The invention described herein relates to the surprising discovery that intranasal administration of a Proteosome-based MV vaccine (including a Proteosome:liposaccharide (LPS)-MV vaccine) can stimulate both a mucosal response in the respiratory tract as well as a systemic antibody response. Moreover, administration of the vaccine compositions described herein in animals, including mice and juvenile rhesus macaques, indicates that the compositions can be safely delivered to a host or subject without any observed toxic or adverse effect. Discussed in more detail herein are immunogenic compositions comprising Proteosomes and one or more measles virus antigens, which are suitable for therapeutic uses such as treating or preventing a measles infection, and methods for preparing the same.

The present invention provides therapeutic compositions comprising one or more measles antigens formulated with a Proteosome-based adjuvant, which compositions can be used as a vaccine to elicit a protective immune response. By way of background, a live attenuated measles vaccine is widely used, and the recommended age of immunization has varied from 6 to 15 months but is still an area under discussion (Volti et al., Eur. J. Epidemiol. 9:311, 1993). Although a respiratory route of immunization has been advocated for younger infants, such attempts have proven either unsuccessful or impractical (Khanum et al., Lancet 1:150, 1987) because of interference from neutralizing maternal antibodies (Markowitz et al., supra). Moreover, drawbacks for the use of the current live measles vaccine include lack of protection at mucosal surfaces where the virus first enters and replicates, low thermal stability of the vaccine, the need for reconstitution prior to injection, and the risk of contamination of injection devices, and unwanted side effects or complications that occur after immunization.

In the present description, any concentration range, percentage range, or integer range is to be understood to include the value of any integer within the recited range and, when appropriate, fractions thereof (such as one tenth and one hundredth of an integer), unless otherwise indicated. As used herein, “about” or “comprising essentially of” mean±15%. The use of the alternative (e.g., “or”) should be understood to mean one, both, or any combination thereof of the alternatives. As used herein, the use of an indefinite article, such as “a” or “an”, should be understood to refer to the singular and the plural of a noun or noun phrase. In addition, it should be understood that the individual compositions, formulations, or compounds, or groups of compositions, formulations, or compounds, derived from the various components or combinations of the composition or sequences, structures, and substituents described herein are disclosed by the present application to the same extent as if each composition or compound or group of compositions or compounds was set forth individually. Thus, selection of particular sequences, structures, or substituents is within the scope of the present invention.

### Measles Virus Polypeptide Immunogens

The present invention is directed generally to the use of measles virus (MV) polypeptide immunogens, including H protein, F protein, M protein, N protein, L protein, P protein, or fragments thereof, including fusions to other polypeptides (e.g., a hydrophobic amino acid sequence) or other modifications (e.g., addition of a lipid or glycosylation). The immunogenic MV polypeptides may comprise any portion of such polypeptides that have at least one epitope capable of eliciting a protective immune response (cellular or humoral) against MV infection. Immunogenic polypeptides of the instant invention may also be arranged or combined in a linear form, and each immunogen may or may not be reiterated, wherein the reiteration may occur once or multiple times. In addition, a plurality of different MV immunogenic polypeptides (e.g., different H protein, F protein, or N protein variants, or fragments thereof) can be selected and mixed or combined into a cocktail composition to provide a multivalent vaccine for use in eliciting a protective immune response. Also contemplated are methods for treating or preventing an MV infection or eliciting an immune response using MV polypeptide immunogens or fragments thereof, or a combination of polypeptides (including fusion proteins).

MV polypeptide immunogens or fragments thereof can be prepared from a variety of biological sources, such as tissues of an infected subject or cultured cell lines. Primary isolation of MV may be from, for example, peripheral blood cells or from respiratory secretions. Preferably, the isolated MV are amplified on primary cell cultures (such as human blood, lung, conjunctiva, kidney, intestine, amnion, skin, muscle, thymic stroma, foreskin, or uterus cells, or monkey kidney or testis cells) or on established cell lines (such as Vero, KB, CV-1, BSC-1, B95-8, WI-38, MRC-5, Hep-2, HeLa, or A549). More preferably, MV polypeptide immunogens or fragments thereof are prepared from an established MV vaccine strain, which are known in the art or are later established in the art. In one preferred embodiment, the MV polypeptide immunogens or fragments thereof are prepared from a Moraten strain, Shwarz strain, Zagreb strain, or Edmonston strain.

In a certain embodiment, the MV polypeptide immunogens or fragments thereof are isolated from intact viral particles. As used herein, the term “isolated” means that the material is removed from its original or natural environment. For example, a naturally occurring nucleic acid molecule or polypeptide present in a living animal or cell, or virus is not isolated, but the same nucleic acid molecule or polypeptide is isolated when separated from some or all of the co-existing materials in the natural system. The nucleic acid molecules, for example, could be part of a vector and/or such nucleic acids or polypeptides could be part of a composition and still be isolated in that such vector or composition is not part of its natural environment. In other embodiments, the MV polypeptide immunogens or fragments thereof may be either partially purified or purified to homogeneity.

As described herein and as is known in the art, a variety of methods may be used to isolate or purify the MV polypeptide immunogens or fragments thereof of the instant invention. MV can be propagated on a cell line of choice, such as Vero cells (African green monkey kidney cells) or CV-1, and the viral particles may be partially or substantially separated from the mammalian cells. For example, a crude extract of MV polypeptide immunogens or fragments thereof can be prepared from infected cells that are subjected to at least one freeze-thaw cycle, centrifuged to remove cells debris, filtered, and the viral particles can be isolated by ultracentrifugation, sonicated, and resuspended in a pharmaceutically acceptable diluent (such as phosphate buffered saline, PBS) (see Example 4). Alternatively or in addition, the MV polypeptide immunogens or fragments thereof can be isolated or purified using a detergent extraction or sucrose density gradient centrifugation to obtain quantifiable amounts of the MV immunogens. As used herein, a “measles split antigen” preparation refers to the separation, isolation, or purification of MV polypeptides from intact measles virus particles. In one preferred embodiment, the MV polypeptide immunogens or fragments thereof comprise a measles split antigen, which may be prepared by way of, for example, detergent solubilization.

The present invention further refers to certain formulations containing one or more viral antigens, wherein the viral antigens may be a part of compositions or components known as lipid rafts. As described herein and is known in the art, such lipid rafts may represent biologically relevant membranes (host cell or virus) enriched for specific viral antigens. Such lipid rafts may be dissociated by treatment with certain detergents, such as octyl glucoside or methyl 13 cyclodextrin, to further modify a vaccine formulation. Thus, lipid raft isolation may be used to enrich for specific desired antigens, or used to aid in formulating a vaccine. The presence or absence of lipid rafts may affect, for example, stability of the immunogen or an immunological outcome.

The present invention further provides methods for producing synthetic MV polypeptide immunogens, including fusion proteins. The immunogenic polypeptide components may be synthesized by standard chemical methods, including synthesis by automated procedure. In general, immunogenic polypeptides or peptides are synthesized based on the standard solid-phase Fmoc protection strategy with HATU as the coupling agent. The immunogenic peptide can be cleaved from the solid-phase resin with trifluoroacetic acid containing appropriate scavengers, which also deprotects side chain functional groups. Crude immunogenic peptide may be further purified using preparative reverse phase chromatography. Other purification methods, such as partition chromatography, gel filtration, gel electrophoresis, ion-exchange chromatography, or other methods practiced by a skilled artisan may be used. Other synthesis techniques known in the art may be employed to produce similar immunogenic peptides, such as the tBoc protection strategy, use of different coupling reagents, and the like. In addition, any naturally occurring amino acid or derivative thereof may be used, including D- or L-amino acids and combinations thereof.

As described herein, the MV polypeptide immunogens or fragments thereof or may be recombinant, wherein a desired MV immunogen is expressed from a polynucleotide that is operatively linked to an expression control sequence (e.g., promoter, enhancer) in a recombinant nucleic acid expression construct. For example, host cells (such as baculovirus and mammalian cell lines) containing H or F or N protein immunogen-encoding nucleic acid expression constructs can be cultured to produce recombinant H or F or N protein immunogens, or fragments thereof (see, e.g., Pütz et al., Intl. J. Parasitol. 33:525 (2003) and references cited therein; see generally Sambrook et al., (2001), supra).

### Vaccine Adjuvants—Proteosomes (“Projuvant” and OMP-LPS)

The invention also relates to immunogenic compositions that contain one or more MV antigen and an additional component to aid or otherwise cooperate in eliciting an immune response, such as an adjuvant. As set forth above, the current live attenuated measles vaccine is poorly immunogenic in children under 9 months of age due to persisting neutralizing maternal antibodies and an immature infant immune system. Drawbacks related to the use of the live attenuated measles vaccine, particularly in third world countries, include lack of thermal stability during storage, which may be an issue in countries with unstable power supplies, and the route of administration is presently by injection, which may lead to transmission of other diseases if the injection is performed in an unsafe manner. Despite the multiplicity of efforts to formulate successful MV vaccines, a need remains for effective compositions to immunize individuals in need thereof, particularly against infection by measles.

An alternative to a live attenuated measles vaccine is an MV subunit vaccine as provided by the instant invention, such as a formulation comprising a split measles antigen preparation and a Proteosome-based adjuvant, as described herein. To maximize the effectiveness of a subunit MV vaccine, the MV antigens may be combined with a potent immunostimulant or adjuvant. Exemplary adjuvants include alum (aluminum hydroxide, REHYDRAGEL®), aluminum phosphate, Proteosome adjuvant (see, e.g., U.S. Pat. Nos. 5,726,292 and 5,985,284, and U.S. Patent Application Publication No. 2001/0053368), virosomes, liposomes with and without Lipid A, Detox (Ribi/Corixa), MF59, or other oil and water emulsions type adjuvants, such as nanoemulsions (see, e.g., U.S. Pat. No. 5,716,637) or submicron emulsions (see, e.g., U.S. Pat. No. 5,961,970), and Freund's complete and incomplete adjuvant. A particularly preferred adjuvant is a Proteosome.

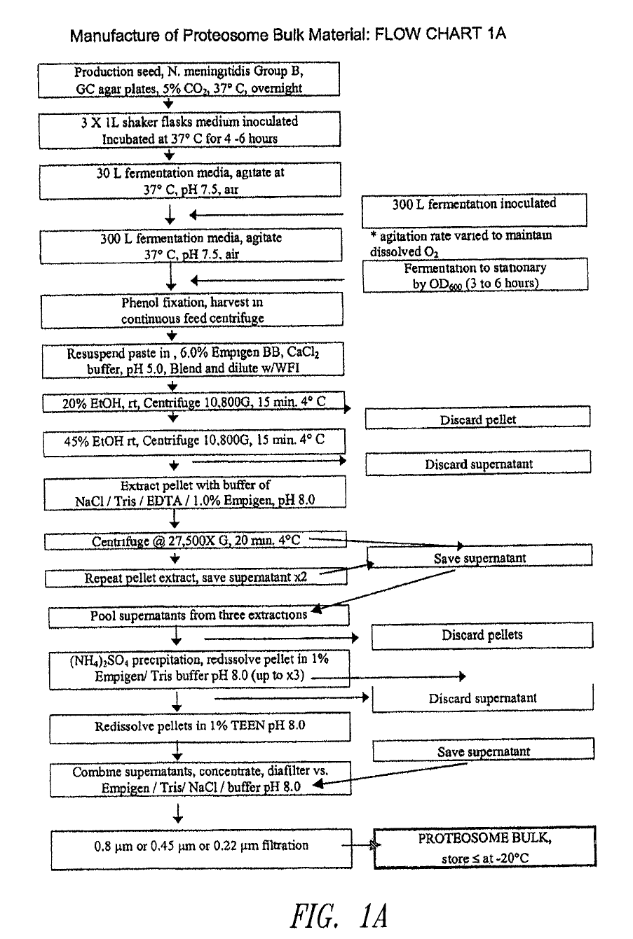

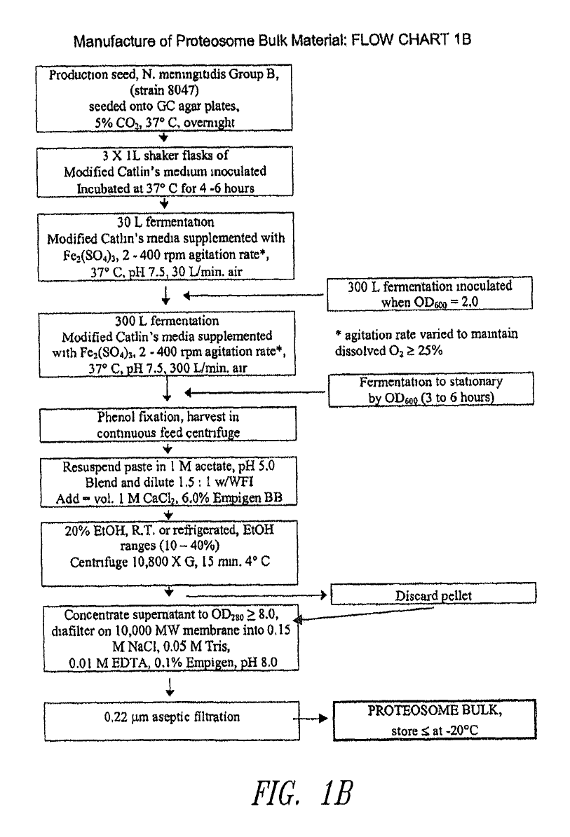

Proteosomes are comprised of outer membrane proteins (OMP) from Neisseria species typically, but can be derived from other Gram-negative bacteria (see, e.g., Lowell et al., J. Exp. Med. 167:658, 1988; Lowell et al., Science 240:800, 1988; Lynch et al., Biophys. J 45:104, 1984; U.S. Pat. No. 5,726,292; U.S. Pat. No. 4,707,543). Proteosomes have the capability to auto-assemble into vesicle or vesicle-like OMP clusters of 20-800 nm, and to noncovalently incorporate, coordinate, associate, or otherwise cooperate with protein antigens (Ag), particularly antigens that have a hydrophobic moiety. Proteosomes are hydrophobic and safe for human use, and comparable in size to certain viruses. By way of background, and not wishing to be bound by theory, mixing of Proteosomes with a protein (e.g., antigen) provides a composition comprising non-covalent association or coordination between the antigen and Proteosomes, which association or coordination forms when solubilizing detergent is selectively removed or reduced, for example, by dialysis. As used herein, “Proteosome” refers to preparations of outer membrane proteins (OMPs) from Gram-negative bacteria, such as Neisseria species (see, e.g., Lowell et al., J. Exp. Med. 167:658, 1988; Lowell et al., Science 240:800, 1988; Lynch et al., Biophys. J 45:104, 1984; Lowell, in “New Generation Vaccines” 2nd ed., Marcel Dekker, Inc., New York, Basil, Hong Kong, pages 193, 1997; U.S. Pat. No. 5,726,292; U.S. Pat. No. 5,985,284; U.S. Pat. No. 4,707,543), which are useful as a carrier or an adjuvant for immunogens, such as MV antigens. Proteosomes may be prepared as described in the art or as described herein (see flowcharts of FIGS. 1A and 1B).

Any preparation method that results in the outer membrane protein component in vesicular or vesicle-like form, including molten globular-like OMP compositions of one or more OMP, is included within the definition of “Proteosome.” In one embodiment, the Proteosomes are from Neisseria species, and more preferably from Neisseria meningitidis. In certain embodiments, Proteosomes are not a carrier but are an adjuvant. As used herein, a Proteosome that is an adjuvant may be referred to as a “projuvant.” In certain other embodiments, Proteosomes may be an adjuvant and an antigen delivery composition. In a preferred embodiment, an MV immunogenic composition of the instant invention comprises one or more MV antigens (i.e., MV immunogens or fragments thereof) as described herein and an adjuvant, wherein the adjuvant comprises a projuvant (i.e., Proteosome) and wherein at least one of the measles antigens is H protein. In another embodiment, this formulation comprises one or more measles virus antigens that include an F protein and an H protein. As described herein, the MV antigens can be from a recombinant source or comprise a measles split antigen. Preferably, the measles split antigen is obtained from a vaccine strain, such as a Moraten strain, Shwarz strain, Zagreb strain, or Edmonston strain.

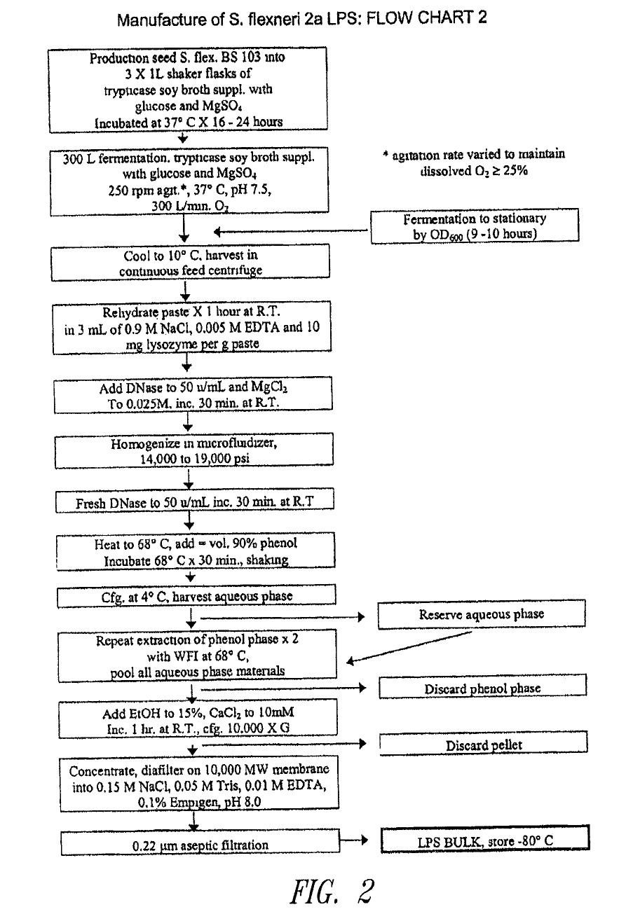

In certain embodiments, the invention provides an immunogenic composition that further comprises an immunostimulant, such as a liposaccharide. That is, the adjuvant may be prepared to include an additional immunostimulant. For example, the projuvant may be mixed as described herein with a liposaccharide to provide an OMP-LPS adjuvant. Thus, the OMP-LPS adjuvant can be comprised of two basic components. The first component is an outer membrane protein preparation of Proteosomes (i.e., projuvant) prepared from Gram-negative bacteria, such as Neisseria meningitidis. The second component is a preparation of liposaccharide. As used herein, “liposaccharide” refers to native or modified lipopolysaccharide or lipooligosaccharide (collectively, also referred to as LPS) derived from Gram-negative bacteria, such as Shigella flexneri or Plesiomonas shigelloides, or other Gram-negative bacteria (including Alcaligenes, Bacteroides, Bordetella, Borrellia, Brucella, Campylobacter, Chlamydia, Citrobacter, Edwardsiella, Ehrlicha, Enterobacter, Escherichia, Francisella, Fusobacterium, Gardnerella, Hemophillus, Helicobacter, Klebsiella, Legionella, Leptospira (including Leptospira interrogans), Moraxella, Morganella, Neiserria, Pasteurella, Proteus, Providencia, other Plesiomonas, Porphyromonas (including Porphyromonas gingivalis), Prevotella, Pseudomonas, Rickettsia, Salmonella, Serratia, other Shigella, Spirillum, Veillonella, Vibrio, or Yersinia species). The liposaccharide may be in a detoxified form (i.e., having the Lipid A core removed) or may be in a form that has not been detoxified. The liposaccharide may be prepared as described in the flowchart of FIG. 2 (see also, e.g., U.S. Patent Application Publication No. 2003/004442). It is also contemplated that the second component may include a lipid, glycolipid, glycoprotein, small molecule, or the like.

Proteosome:LPS or Protollin™ or IVX or IVX-908 as used herein refers to preparations of projuvant admixed as described herein with at least one kind of liposaccharide to provide an OMP-LPS composition (which can function as an immunostimulatory composition). Thus, the OMP-LPS adjuvant can be comprised, for example, of two of the basic components of IVX-908, which include (1) an outer membrane protein preparation that is a Proteosome (i.e., Projuvant) prepared from Gram-negative bacteria, such as Neisseria meningitidis, and (2) a preparation of one or more liposaccharides.

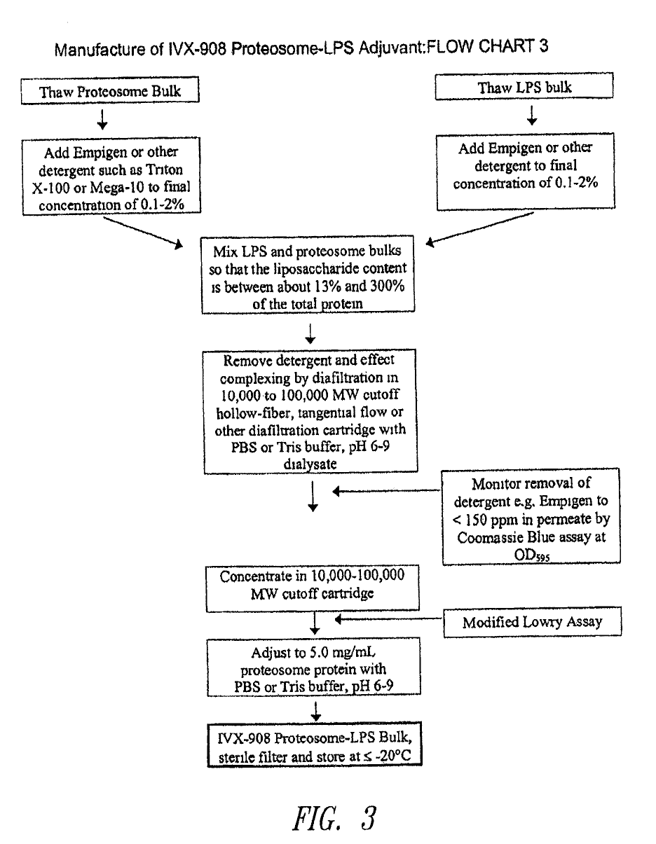

As described herein, the two components of an OMP-LPS adjuvant may be formulated at specific initial ratios (see flowchart of FIG. 3) to optimize interaction between the components resulting in stable association and formulation of the components for use in the preparation of an MV immunogenic composition described herein. The process generally involves the mixing of components in a selected detergent solution (e.g., Empigen® BB (n-Dodecyl-N,N-dimethylglycine), Triton® X-100 (octyl phenol ethoxylate), or Mega-10 (n-Decanoyl-N-methylglucamide)) and then effecting complexing of the OMP and LPS components while reducing the amount of detergent to a predetermined, preferred concentration, by dialysis or, preferably, by diafiltration/ultrafiltration methodologies. Mixing, co-precipitation, or lyophilization of the two components may also be used to effect an adequate and stable association or formulation. In a preferred embodiment, an MV immunogenic composition of the instant invention comprises one or more MV antigens (i.e., MV immunogens or fragments thereof) as described herein and an adjuvant, wherein the adjuvant comprises a projuvant (i.e., Proteosome) and liposaccharide, wherein at least one of the measles antigens is H protein. In another embodiment, this formulation comprises one or more measles virus antigens that include an F protein and an H protein. As described herein, the MV antigens can be from a recombinant source or comprise a measles split antigen. Preferably, the measles split antigen is obtained from a vaccine strain, such as a Moraten strain, Shwarz strain, Zagreb strain, or Edmonston strain.

In the preferred embodiment, the final liposaccharide content by weight as a percentage of the total Proteosome protein can be in a range from about 10% to about 500%, in a range from about 20% to about 200%, or in a range from about 30% to about 150%. In one preferred embodiment the adjuvant composition comprising Proteosomes is prepared from Neisseria meningitidis and the liposaccharide is prepared from Shigella flexneri or Plesiomonas shigelloides, and the final liposaccharide content is between 50% to 150% of the total Proteosome protein by weight. In another embodiment, Proteosomes are prepared with endogenous lipooligosaccharide (LOS) content ranging from about 0.5% up to about 5% of total OMP. Another embodiment of the instant invention provides Proteosomes with endogenous liposaccharide in a range from about 12% to about 25%, and in a preferred embodiment between about 15% and about 20% of total OMP. The instant invention also provides a composition containing liposaccharide derived from any Gram-negative bacterial species, which may be from the same Gram-negative bacterial species that is the source of Proteosomes or is a different bacterial species.

### Immunogenic Compositions and Methods of Use

The immunogenic compositions described herein that contain one or more MV immunogens, which can be used to elicit an immune response, such as a protective immune response. The invention provides methods for treating and preventing MV infections by administering to a subject one or more MV immunogens or fragments thereof, fusion protein, multivalent immunogen, or a mixture of such immunogens at a dose sufficient to elicit an immune response (cellular and/or humoral) specific for MV (which may be a protective immune response), as described herein. MV polypeptide immunogens and variants thereof, or a cocktail of such immunogens are preferably part of a composition comprising an adjuvant, such as projuvant or OMP-LPS, when used in the methods of the present invention. In one embodiment, the immunogenic compositions of the instant invention may further comprise one or more additional microbial antigens, such as viral antigens, bacterial antigens, parasitic antigens, or a combination thereof. For example, the MV immunogenic composition may also include antigens for rubella and mumps.

The immunogenic compositions may further include a pharmaceutically acceptable vehicle, carrier, diluent, or excipient, in addition to one or more MV immunogen or fragment thereof and, optionally, other components. For example, pharmaceutically acceptable carriers or other components suitable for use with an immunogenic composition of this invention include a thickening agent, a buffering agent, a solvent, a humectant, a preservative, a chelating agent, an additional adjuvant, and the like, and combinations thereof.

In addition, the pharmaceutical composition of the instant invention may further include a diluent such as water or phosphate buffered saline (PBS). Preferably, diluent is PBS with a final phosphate concentration range from about 0.1 mM to about 1 M, more preferably from about 0.5 mM to about 500 mM, even more preferably from about 1 mM to about 50 mM, and most preferably from about 2.5 mM to about 10 mM; and the final salt concentration ranges from about 100 mM to about 200 mM and most preferably from about 125 mM to about 175 mM. Preferably, the final PBS concentration is about 5 mM phosphate and about 150 mM salt (such as NaCl). In certain embodiments, any of the aforementioned immunogenic compositions comprising a cocktail of MV immunogens or MV split antigen and an adjuvant (such as projuvant or OMP-LPS) of the instant invention are preferably sterile.

The compositions can be sterile either by preparing them under an aseptic environment or they can be terminally sterilized using methods available in the art. Many pharmaceuticals are manufactured to be sterile and this criterion is defined by the USP XXII <1211>. Sterilization in this embodiment may be accomplished by a number of means accepted in the industry and listed in the USP XXII <1211>, including gas sterilization, ionizing radiation or filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. When appropriate, filtration may be accomplished using a filter with suitable pore size, for example 0.22 μm and of a suitable material, for instance Teflon® (fluorine-containing polymer). The term “USP” refers to U.S. Pharmacopeia (see www.usp.org; Rockville, Md.). Due to the fact that Proteosomes or OMP-LPS result in particles small enough that the immunogenic compositions of the invention can be filtered through a 0.8μ filter, a 0.45μ filter, or a 0.2μ filter. Thus, in preferred embodiments the MV immunogenic compositions of this invention are sterilized by filtration. This is highly advantageous as it is desirable to eliminate any complications by virtue of the presence of such contaminants.

The present invention also pertains to methods for treating or preventing a measles infection, comprising administering to a subject in need thereof an immunogenic composition comprising an adjuvant and one or more measles virus antigens, wherein the adjuvant comprises either Proteosomes or OMP-LPS, and at least one of the measles antigens is an H protein. In another embodiment, the immunogenic compositions of this invention may be used to elicit an immune response (cellular or humoral or both, which may favor a Type 1 or Type 2 cellular response). A subject suitable for treatment or for eliciting an immune response with a MV immunogen formulation may be identified by well-established indicators of risk for developing a disease or well-established hallmarks of an existing disease. Infections that may be treated with a MV immunogen disclosed herein include infections caused by or due to MV, whether the infection is primary, secondary, opportunistic, or the like. Examples of MV include any antigenic variant of these viruses.

Methods for preparing the immunogenic compositions of the instant invention are described herein and are known in art (see, e.g., U.S. Patent Application Publications Nos. 2001/0053368 and 2003/0044425). The antigen(s) and adjuvant are formulated at specific initial ratios to optimize interaction (or cooperation) between the components resulting in non-covalent association (or non-specific juxtaposition) of a significant portion of the two components with each other. For example, a mixture of at least one MV polypeptide antigen with a Proteosome (projuvant) or OMP-LPS is prepared in the presence of detergent, and reduction or removal of the detergent from the mixture by diafiltration/ultrafiltration leads to association (or coordination) of the antigens with the adjuvant (see FIG. 3). In preferred embodiments, the Proteosome to viral antigen ratio in the mixture is greater than 1:1, preferably greater than 2:1, more preferably greater than 3:1 and more preferably greater than 4:1. The ratio can be as high as 8:1 or higher. Alternatively, the ratio of Proteosome to viral antigen in the mixture is 1:1, 1:2, 1:3, 1:4, or 1:8. The detergent-based solutions of the two components may contain the same detergent or different detergents, and more than one detergent may be present in the mixture subjected to ultrafiltration/diafiltration. Suitable detergents include Triton® Empigen® BB, and Mega-10. Other detergents can also be used. The detergents serve to solubilize the components used to prepare the composition. The use of a mixture of detergents may be particularly advantageous. This mixture is, of course, removed or the concentration is reduced by diafiltration/ultrafiltration prior to final formulation.

The immunogenic compositions that contain one or more MV antigens and a Proteosome-based adjuvant described herein may be in any form that allows for the composition to be administered to a subject, such as a human or non-human animal (e.g., a non-human primate, or rodent, for example, a mouse or rat). For example, such immunogenic compositions may be prepared and administered as a liquid solution or prepared as a solid form (e.g., lyophilized), which may be administered in solid form, or resuspended in a solution in conjunction with administration. The MV immunogenic polypeptide compositions are formulated to allow the active ingredients contained therein to be bioavailable upon administration of the composition to a subject (or patient) or bioavailable via slow release. Compositions that will be administered to a subject or patient take the form of one or more dosage units. For example, a drop may be a single dosage unit, and a container of one or more compounds of the invention in aerosol form may hold a plurality of dosage units. In certain preferred embodiments, any of the aforementioned pharmaceutical compositions comprising a MV immunogen or cocktail of immunogens of the invention are in a container, preferably in a sterile container. The design of a particular protocol for administration, including dose level, time of dosing, number of doses, time periods between dosing are determined by optimizing such procedures using routine methods well known to those having ordinary skill in the art.

In one embodiment, the immunogenic composition is administered nasally. Other typical routes of administration include enteral, parenteral, transdermal/transmucosal, nasal, and inhalation. The term “enteral”, as used herein, is a route of administration in which the immunogenic composition is absorbed through the gastrointestinal tract or oral mucosa, including oral, rectal, and sublingual. The term “parenteral”, as used herein, describes administration routes that bypass the gastrointestinal tract, including intraarterial, intradermal, intramuscular, intranasal, intraocular, intraperitoneal, intravenous, subcutaneous, submucosal, and intravaginal injection or infusion techniques. The term “transdermal/transmucosal”, as used herein, is a route of administration in which the immunogenic composition is administered through or by way of the skin, including topical. The terms “nasal” and “inhalation” encompass techniques of administration in which an immunogenic composition is introduced into the pulmonary tree, including intrapulmonary or transpulmonary. A composition may be adminstered as an aerosol by a mechanism known in the art, such as by a mechanical apparatus, for example, a nebulizer, whereby the aerosolized composition is delivered to the upper and lower respiratory tract. Preferably, the immunogenic compositions described herein are administered nasally (intranasally).

Furthermore, the immunogenic compositions disclosed herein can be used to enhance immunity, or as a follow-on immunization, when given together with another vaccine, such as a live attenuated measles vaccine. For example, compositions comprising one or more MV polypeptide immunogens with projuvant or OMP-LPS may be used as a priming immunization or as a boosting immunization (by mucosal or parenteral routes) prior to or subsequent to administering a live attenuated measles vaccine.

In another embodiment, for treating or preventing a measles infection and/or for eliciting an immune response, a subject receives at least one, two, or three priming immunizations with a DNA vaccine followed by a boosting immunization with the compositions disclosed herein comprising one or more MV polypeptide immunogens with projuvant or OMP-LPS. The DNA vaccine comprises one or more recombinant expression constructs that contain a polynucleotide sequence encoding a measles virus polypeptide, or fragment thereof, and that is operatively linked to a promoter sequence (see, e.g., Fennelly et al., J. Immunol. 162:1603-10 (1999); Pasetti et al., J. Virol. 77:5209-17 (2003)). The polynucleotide may encode at least one measles virus polypeptide, for example H protein, may encode at least two measles virus polypeptides (i.e., a bicistronic polynucleotide), for example, H protein and F protein, or may encode at three, four, or five or more measles virus polypeptides (i.e., a polycistronic polynucleotide). The DNA vaccine may comprise two or more recombinant expression constructs, for example, wherein each construct comprises a polynucleotide containing a promoter that is operatively linked to a polynucleotide sequence that encodes at least one measles virus polypeptide, or fragment thereof.

Recombinant polynucleotide expression constructs may be prepared according to methods known to persons skilled in the molecular biology art. Cloning and expression vectors for use with prokaryotic and eukaryotic hosts are described, for example, in Sambrook et al., Molecular Cloning: A Laboratory Manual, Third Edition, Cold Spring Harbor, N.Y., (2001), and may include plasmids, cosmids, shuttle vectors, viral vectors, and vectors comprising a chromosomal origin of replication as disclosed therein. Recombinant expression constructs also comprise expression control sequences (regulatory sequences) that allow expression of a polypeptide of interest in a host cell, including one or more promoter sequences. (e.g., lac, tac, trc, ara, trp, λ phage, T7 phage, T5 phage promoter, CMV, immediate early, HSV thymidine kinase, early and late SV40, LTRs from retrovirus, and mouse metallothionein-I), enhancer sequences, operator sequences (e.g., lacO), and the like.

Generally, recombinant expression vectors will include origins of replication and selectable markers permitting transformation of the host cell, and a promoter derived from a highly-expressed gene to direct transcription of a downstream structural sequence. The heterologous structural sequence is assembled in appropriate phase with translation initiation and termination sequences. In preferred embodiments the constructs are included in compositions that are administered in vivo. Such vectors and constructs include chromosomal; nonchromosomal; and synthetic DNA sequences, e.g., derivatives of SV40; bacterial plasmids; phage DNA; yeast plasmids; vectors derived from combinations of plasmids; and phage DNA; viral DNA, such as vaccinia, adenovirus, fowl pox virus, and pseudorabies; or replication deficient retroviruses as described below. However, any other vector may be used for preparation of a recombinant expression construct, and in preferred embodiments such a vector will be replicable and viable in the host (subject).

In one embodiment, the DNA vaccine is prepared by introducing a recombinant expression vector into bacteria, which bacteria are then administered to a subject. For example, a recombinant expression vector that comprises a polynucleotide encoding one or more measles virus polypeptides, or fragment thereof, may be introduced (e.g., by transfection, electroporation, or transformation) into a strain of Shigella flexneri (see, e.g., Fennelly et al., supra; Pasetti et al., supra). The bacteria may then be prepared for administration to a subject according to methods practiced by skilled artisans for delivery of such DNA vaccines. The DNA vaccine may be delivered intranasally, intramuscularly, intradermally, parenterally, by inhalation, or by any other route and method in the art that provides the vaccine to the subject in a manner such that the encoded My polypeptides are expressed.

Preferably, the MV immunogenic compositions described herein will induce specific anti-MV immune responses, including one or more of a systemic humoral response, a mucosal immune response, and cell-mediated immunity (CMI). A systemic humoral immune response is indicated by the presence of specific anti-measles antigen IgG antibodies or other classes of immunoglobulin in serum, the protective or therapeutic effect of which may be determined in functional assays, including hemagglutination inhibition (HI) assays. Induction of a response measured by HI is useful because the presence of an immunoglobulin in a biological sample from an immunized subject that inhibits hemagglutination is believed to correlate with protection against MV in humans. A mucosal immune response includes production of mucosal antibodies, including IgA in mucosal secretions such as those collected from the respiratory tract, including the nasopharynx and lungs. Not wishing to be bound by theory, the mucosal immune response system likely provides the initial immunological barrier against MV infection, and IgA that is predominant in a mucosal humoral response mediates the defense functions. Analysis of anti-MV IgA antibodies in vitro suggests that the anti-MV immune response prevents virus entry, interrupts virus replication, and/or disrupts transport of virus across the epithelium (see, e.g., Lamm, Annu. Rev. Microbiol. 51:311-40 (1997); Yan et al., J. Virol. 76:430-35 (2002)).

Cell populations that comprise the mucosal barrier can respond to signals that can reach local or distant sites within the body (Svanborg et al., Curr. Opin. Microbiol. 2:99-105 (1999)). According to non-limiting theory, toll-like receptors (TLRs) are key components of the innate immune system, and it likely that IVX908-MV act through the TLR system because PorB, the major N. meningitidis Omp protein in IVX908, binds TLR-2 (Massari et al., J. Immunol. 168:1533-37 (2002)). LPS activates TLR-4 (Takeda et al., Annu. Rev. Immunol. 21:335-76 (2003)), and the H protein of measles can also bind TLR-2 (Bieback et al., J. Virol. 76:8729-36)). TLR engagement results in the production of proinflammatory cytokines (e.g., IFN-γ, TNF-α, and IL-12) and the upregulation of costimulatory molecules on antigen-presenting cells. The activated innate response directs the effective adaptive immune response.

Cell-mediated immunity (CMI) includes the switch or decrease from a higher or predominant T_{H}2 response to result in mixed, balanced, increased or predominant T_{H}1 response, for example, as determined by induction of cytokine expression, such as IFN-γ, without comparable increases in induction of certain T_{H}2 cytokines, such as IL-5 which levels may, for example, be maintained, decreased, or absent. Such T_{H}1 responses are predictive of the induction of other CMI associated responses, such as development of cytotoxic T cells (CTLs), which are indicative of T_{H}1 immunity.

The presence of measles specific antibodies in a biological sample from a subject, including sera, nasal lavage, and/or lung lavage, may be determined by any one of numerous immunoassays practiced in the art. Such immunoassays include but are not limited to ELISA, immunoblot, radioimmunoassay, and Ochterlony. Determining the functional activity of measles-specific antibodies may also be determined according to methods described herein and known in the art, such as plaque reduction neutralization assays, hemagglutination inhibition assays, and assays that determine the presence of opsonizing antibodies.

The capability of a MV vaccine composition, such as Proteosome:MV and/or a IVX908-MV composition described herein to elicit a specific immune response against MV and/or to prevent a measles virus infection or treat a measles virus infection in a subject may be determined in animal models that are described herein and known and accepted in the art. For example, a murine model or a non-human primate model, such as a rhesus macaque model may be used. One, two, three or more doses of a MV vaccine composition may be administered to the animals as primary and boosting immunizations or as one or more boosting immunizations following a primary or priming immunization with a different vaccine, such as an attenuated measles vaccine or a measles DNA vaccine. Preferably, the MV vaccine is delivered to the animals in a similar manner to the delivery method that may be used for administering the vaccines to humans, such as intranasally. The immune response in the animals may be assessed by determining the presence of immunoglobulins that specifically bind to and/or exhibit a function that indicates that the response is therapeutic or protective. For example, the immunoglobulins, particularly IgG and IgA antibodies, may be sampled to determine when and whether a specific immune response has occurred. Examplary assays described herein and known in the art include immunoassays (e.g., ELISA and immunoblot); determination of measles virus-specific cytokine production (e.g., IFNγ, IL4, IL5); and plaque reduction neutralization (PRN) assays. PRN values are particularly useful for characterizing the immune response and evaluating whether the animal when challenged with measles virus will be protected from developing sequelae related to measles infection and disease. Seroprotection in humans has been defined as a PRN value greater than 120 (Chen et al., J. Infect. Dis. 162:1036-42 (1990)).

As described herein, adjuvant compositions, including Proteosome compositions and OMP-LPS compositions may also be combined with one or more antigens from one or more microbes (virus, bacteria, parasite, fungus) other than or in addition to measles virus and used for treatment or prevention of other infectious diseases. For example, Proteosome:antigen or IVX908:antigen compositions prepared as described herein may be used for treating or preventing diseases resulting from infection by rubella or mumps viruses. Such immunogenic compositions may also be used for eliciting an immune response that is specific to a virus, such as a rubella or mumps virus. Viral antigens for use in such compositions may be isolated or partially isolated from virus particles, or derived from a cell infected with the virus, or expressed recombinantly according to standard molecular biology methods and then isolated. One or more of the viral antigens may be combined with a Proteosome or OMP-LPS adjuvant according to the methods described herein.

The viral antigens combined with a Proteosome or OMP-LPS adjuvant may be from a single type of virus or may be used in a cocktail, that is, one or more antigens from one virus may be combined with one or more antigens of one or more other viruses. Any of a number of cocktails or combinations may be prepared. For example, one composition may comprise antigens from measles, rubella, and mumps virus, or antigens from measles and rubella viruses, or antigens from measles and mumps viruses, or antigens from mumps and rubella viruses. Any one or more antigens from one or more viruses may then be combined with a Proteosome or OMP-LPS adjuvant. Alternatively, a Proteosome:rubella antigen(s) and/or OMP-LPS:mumps antigen(s) compositions may be combined with a Proteosome:MV or IVX908-MV composition described herein and administered in any combination to a subject in need thereof. Eeach immunogenic composition may be administered separately from another immunogenic composition at different times (and routes). Any of these immunogenic compositions may be used as a primary (initial or priming) immunization and a boosting immunization or may be used as a boosting immunization. An alternative priming (or primary) immunogen may comprise a DNA vaccine containing a polynucleotide that encodes at least one, two, three, four, or more viral polypeptides of a virus to which the subsequent boosting immunization is directed. These DNA vaccines may be prepared by methods described herein and known in the art.

All U.S. patents, U.S. patent application publications, U.S. patent applications, foreign patents, foreign patent applications, and non-patent publications referred to in this specification and/or listed in the Application Data Sheet, are incorporated herein by reference, in their entirety. The following examples are intended to illustrate, and not limit, the invention described herein.

### EXAMPLES

### Example 1

### Preparation of Proteosomes

Immunogens (e.g., measles virus antigens) may be formulated with Proteosomes to form a vaccine composition of the instant invention capable of eliciting a protective immune response in a human or animal subject. Proteosomes are useful as an adjuvant and are comprised of outer membrane proteins purified from Gram-negative bacteria. Methods for preparing Proteosomes are described in, for example, Mallett et al. Infect. Immun. 63:2382, 1995; U.S. Pat. No. 6,476,201 B1; U.S. Patent Application Publication No. 2001/0053368; and U.S. Patent Application Publication No. 2003/0044425. Briefly, a paste of phenol-killed Group B type 2 Neisseria meningitides was extracted with a solution of 6% Empigen® BB (EBB) (Albright and Wilson, Whithaven, Cumbria, UK) in 1 M calcium chloride. The extract was precipitated with ethanol, solubilized in 1% EBB-Tris/EDTA-saline, and then precipitated with ammonium sulfate. The precipitated Proteosomes were re-solubilized in 1% EBB buffer, diafiltered, and stored in a 0.1% EBB buffer at −70° C.

A flow chart of this process, which resulted in Proteosomes having a liposaccharide content of between about 0.5% and about 5%, is shown in Flowchart 1A (FIG. 1A). Proteosomes may also be prepared by omitting the ammonium sulphate precipitation step to shorten the process as desired with resultant Proteosomes having a liposaccharide content of between about 12% and about 25%, and may, depending upon the materials, be between about 15% and about 20%, as shown in Flowchart 1B (FIG. 1B). It should be understood that a person having ordinary skill in the art could adjust methods for preparing formulations comprising Projuvant or OMP-LPS compositions of the instant invention to fit particular characteristics of the vaccine components.

### Example 2

### Preparation of Liposaccharides

The example in Flowchart 2 (FIG. 2) shows the process for the isolation and purification of LPS from S. flexneri or P. shigelloides. This process can similarly be used for preparing LPS from other gram-negative bacteria, including Shigella, Plesiomonas, Escherichia, and Salmonella species. Following growth of bacteria by fermentation in 300 L, the bacteria were sedimented and the cell paste was re-hydrated with 3 mL 0.9M NaCl, 0.005 M EDTA and 10 mg lysozyme per gram of bacterial paste. Lysozyme digestion was allowed to proceed for 1 hour at room temperature. Then 50 U/ml Benzonase® (DNase) in 0.025 M MgCl₂ was added and DNase digestion was allowed to proceed at room temperature for 30 minutes. The suspension was then cracked by passage through a microfluidizer at 14,000 to 19,000 psi. Fresh DNase (50 U/mL) was added, and digestion of the suspension was allowed to proceed for a further 30 min at room temperature. The digested cell suspension was heated to 68° C. in a water bath, an equal volume of 90% phenol (also heated to 68° C.) was added, and then the mixture was incubated with shaking at 68° C. for 30 min. The mixture was centrifuged at 4° C. to separate the aqueous and organic phases. The aqueous phase was harvested and the organic phase was re-extracted with WFI (water for injection) at 68° C. for 30 min. The mixture was centrifuged at 4° C., the second aqueous phase was harvested, and the two harvested aqueous phases were combined. To precipitate nucleic acids, 20% ethanol with 10 mM CaCl₂ was added to the pooled aqueous phases. The mixture was stirred at 4° C. overnight and precipitated nucleic acids were then sedimented by centrifugation at 10,000×g for 30 minutes. The supernatant was harvested, concentrated, and diafiltered using a 30,000 MW hollow fiber cartridge into 0.15M NaCl, 0.05M Tris, 0.01 M EDTA and 0.1% Empigen® BB, pH 8.0. Finally, the LPS was sterile-filtered using a 0.22 μm Millipak® 60 filter unit, aliquoted into sterile storage containers, and frozen at −80° C.

### Example 3

### Preparation and Characterization of Proteosome:Liposaccharide Adjuvant

A Proteosome adjuvant formulation of the instant invention was manufactured by admixing Proteosomes and LPS to allow a presumably non-covalent association. The LPS can be derived from any of a number of gram negative bacteria, such as Shigella, Plesiomonas, Escherichia, or Salmonella species (see Example 2), which is mixed with the Proteosomes of Example 1, as described in Flowchart 3 (FIG. 3). Briefly, Proteosomes and LPS were thawed overnight at 4° C. and adjusted to 1% Empigen® BB in TEEN buffer. The two components were mixed, for 15 minutes at room temperature, at quantities resulting in a final wt/wt ratio of between about 10:1 and about 1:3 of Proteosome:LPS. The Proteosome:LPS mixture was diafiltered on an appropriately sized (e.g., Size 9) 10,000 MWCO hollow fiber cartridge into TNS buffer (0.05 M Tris, 150 mM NaCl pH 8.0). The diafiltration was stopped when Empigen® content in the permeate was <50 ppm (by Empigen® Turbidity Assay or by a Bradford Reagent Assay). The bulk adjuvant (referred to herein as OMP-LPS) was concentrated and adjusted to 5 mg/mL protein (by Lowry assay). Finally, the adjuvant was sterile-filtered using a 0.22 μm Millipak 20 filter unit. The bulk adjuvant was aliquoted into sterile storage containers and frozen.

The OMP-LPS adjuvant was tested for (1) Empigen® (400 ppm) using reverse-phase HPLC; (2) protein content by a Lowry assay; (3) LPS content by measurement of 2-keto-3-deoxyoctonate (KDO) assay. The OMP-LPS composition was further characterized for particle size distribution as determined by quantitative number weighted analysis using a particle seizer (Brookhaven Instruments model 90 plus or similar machine) (10-100 nm). However, the particle size for the complex may increase or modulate with varying (e.g., higher) Proteosome to LPS ratio. Stability of the OMP-LPS composition in the adjuvant formulation should be consistent with the previously demonstrated for S. flexneri LPS vaccine (see U.S. Patent Application Publication No. 2003/0044425). These data demonstrate that OMP-LPS composition was stable at both refrigerated and accelerated temperature (25° C. and 37° C.). Under these conditions, the LPS component of the composition or any statistically significant portion thereof may be complexed with the Proteosome component of the vaccine formulation.

### Example 4

### Preparation of Measles Virus Antigen

Propagation of an attenuated strain of measles virus (MV) used for vaccine purposes in the United States, the Moraten strain, was accomplished by infecting Vero green monkey kidney cells at a multiplicity of infection (MOI) of 0.01-0.001, which infected cells were cultured in a 10 level factory chamber (Nalge® Nunc® International, Rochester, N.Y.). The MOI was low to minimize the generation of defective, interfering particles. Infected cell cultures were monitored until significant cytopathology was detected (e.g., about 3-5 days), at which time infected cell cultures were subjected to one freeze-thaw cycle to disrupt cells. Cell debris was removed by centrifugation at 2100×g for 20 minutes at 4° C. The supernatant containing cell-free MV was recovered and filtered sequentially through a 0.45 μm filter and then a 0.22 μm filter. The filtered supernatant was then ultracentrifuged at 14,000 rpm for two hours at 4° C. The sedimented measles virus particles were resuspended in phosphate buffered saline (PBS) and then subjected to sonication. Protein concentration was determined using a bicinchoninic acid protein assay (Pierce® Biotechnology, Rockford, Ill.) and a standard curve prepared using a mixture of bovine serum albumin fraction V (BSA) and bovine gamma globulin fraction II (BGG).

### Example 5

### Analysis of Measles Virus Antigen Preparation

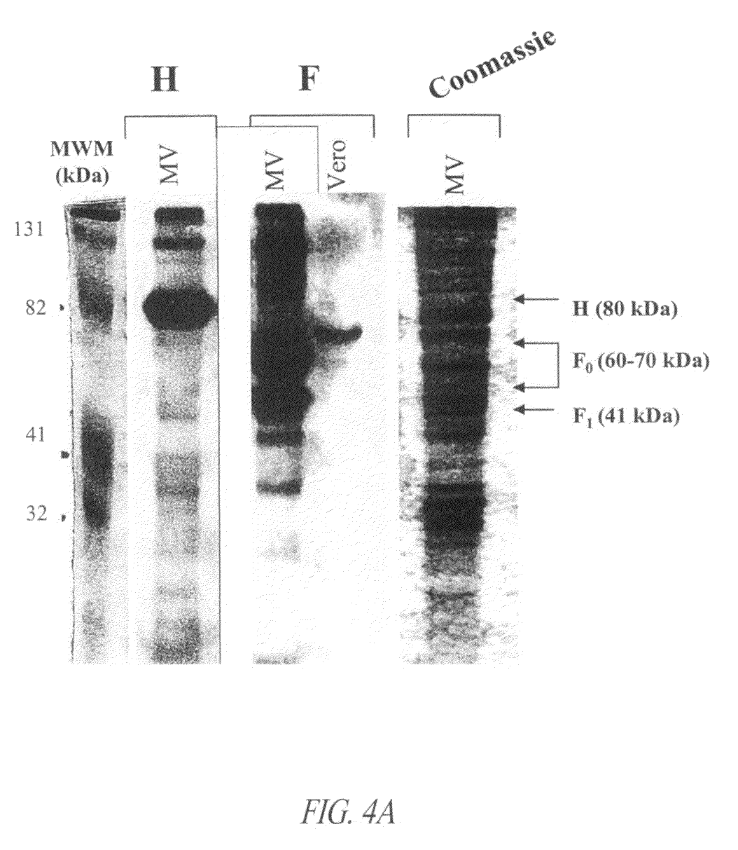

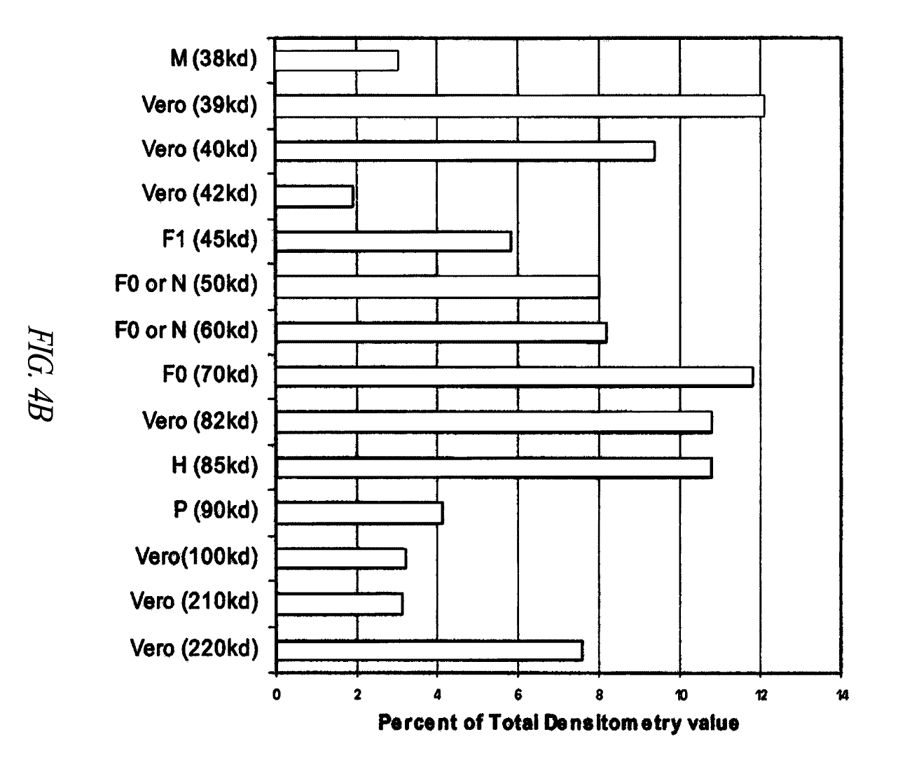

Sodium dodecyl sulfate (SDS)-polyacrylamide gel electrophoresis (SDS-PAGE) was performed to evaluate the presence of MV hemagglutinin (H) protein and fusion (F) protein in the virus preparation (see FIG. 4). Serial dilutions of virus samples were separated by electrophoresis on a 10% polyacrylamide gel and protein bands were visualized by staining with Coomassie® Brilliant Blue G-250 (Kodak®, Rochester, N.Y.). The relative amounts of individual MV antigens detected by Coomassie® Brilliant Blue staining and the amount of H and F protein determined as a proportion of total protein content of the sample was determine by quantitative densitometry using Scion image software. The amount of H protein and F protein in the protein preparation was 44.4%.

In parallel, separated MV samples from another gel were transferred to PVDF membranes for evaluation by immunoblot analysis for MV H and F proteins. After transfer, membranes were blocked with 5% skim milk in PBS containing 0.1% Tween 20 (polyoxyethylenesorbitan monooleate) (PBS-T) and then incubated with monoclonal antibodies capable of detecting MV H or F proteins, room temperature for 60 minutes. Immunoblots were then washed with PBS-T followed by incubation in the presence of goat-anti-mouse-HRP (Jackson Immunoresearch Laboratories) for 60 minutes at room temperature, after which, membranes were incubated with HRP substrate, ECL kit (Amersham® Biosciences); signal was visualized by exposing Immunoblots to X-ray film (Kodak®, Rochester, N.Y.).

Bands corresponding to MV H and F proteins were detected using both Coomassie® Brilliant Blue and immunoblot analysis. For example, a MV H protein band of 80 kDa was detected on superimposed immunoblots and Coomassie® stained gels. The MV F protein detected by immunoblot showed the presence various F protein sizes, which is expected because the Fₒ primary translation product is proteolytically processed into F₁ and F₂ subunits. Two forms of Fₒ of 50-60 kDa can be identified, most likely due to a difference in post-translation glycosylation. The F₁ band was identified as a 41 kDa protein band. Some cross reactivity between Vero cell proteins and the F antibody used in these experiments was detected (see, e.g., Vero cell extract control lanes of FIG. 4A).

### Example 6

### Preparation of Formulations Comprising Proteosome:LPS and Measles Virus Antigen

A formulation of the current invention was prepared by mixing the Proteosome:LPS adjuvant (also referred to herein as OMP-LPS) from Example 3 with the MV antigen from Example 4 in proportions that promote optimal stability and immunological outcomes. In some cases, prior to formulation with the Proteosome:LPS adjuvant, virus, antigen preparations were adjusted to contain 1% detergent (e.g., Empigen® BB or Mega-10), followed by dialysis, and then mixing with Proteosome:LPS adjuvant.

### Example 7

### Preparation of Formulations Comprising Proteosomes and Measles Virus Antigen

A formulation of the current invention was prepared by mixing the Proteosomes from Example 1 with the MV antigen from Example 4 in proportions that promote optimal formulations for stability and immunological outcomes. Prior to formulation with Proteosomes, virus antigen preparations were adjusted to contain 1% detergent (e.g., Empigen® BB or Mega-10), as disclosed in Example 6.

### Example 8

### Analysis of measles virus antigen vaccine formulations

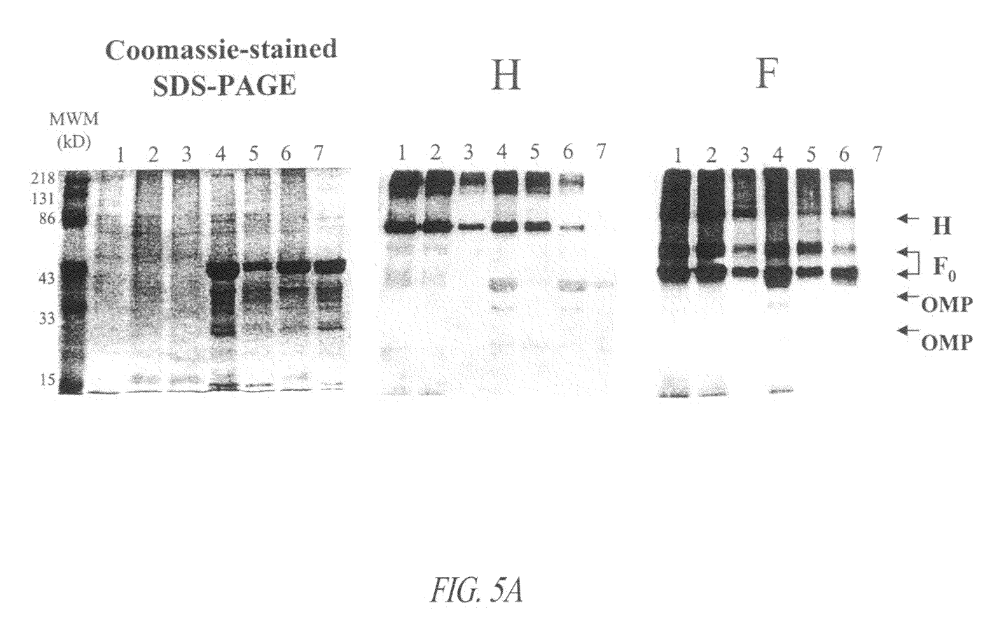

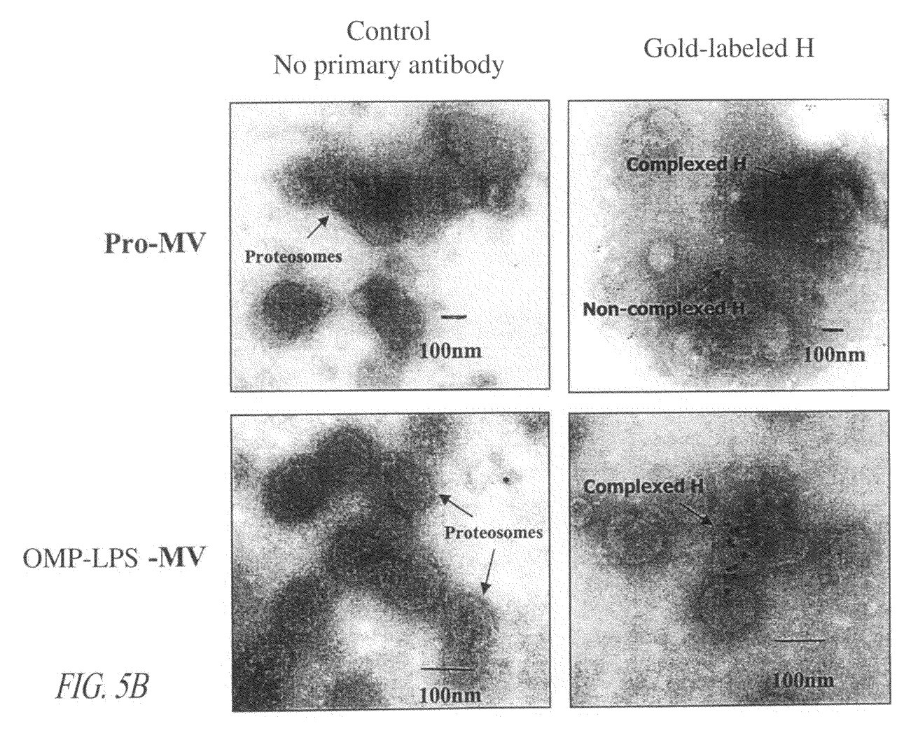

Vaccine formulations were analyzed by SDS-PAGE (Coomassie® Brilliant Blue staining and immunoblot analysis) and by immunogold electron microscopy. Before SDS-PAGE analysis, vaccine formulations were centrifuged at 10,000 rpm for 15 seconds. Soluble (supernatant) and insoluble (pellet) fractions of the vaccine formulations were collected. Insoluble fractions were resuspended in PBS before the addition of sample buffer containing β-mercaptoethanol. In the case of Proteosome formulated MV vaccine compositions, the presence of Proteosome OMPs in the soluble fraction of the vaccine formulation was monitored and served, in these experiments, as an indicator of a successful formulation process. Coomassie® Brilliant Blue staining was used to detect proteins present in soluble and insoluble fractions, and immunoblot analysis was used to confirm the presence of MV H and F proteins in dialyzed preparations. Vaccine formulations of Proteosome with MV antigen, and OMP:LPS with MV antigen, were found to contain detectable amounts of MV H and F proteins (FIG. 5A).

For analysis by electron microscopy (FIG. 5B), vaccine formulation samples were airfuged onto nickel grids for 5 minutes. Grids were immersed in a blocking solution containing 1% bovine serum albumin (BSA) for 5 minutes, washed, and then incubated with monoclonal antibodies (Chemicon® International, Temecula, Calif.) against MV H protein for 60 minutes at room temperature. Grids were then blocked with 1% BSA for 5 minutes, and subsequently incubated with anti-mouse IgG-Gold-10 nm for one hour, washed with PBS, double distilled water and air dried, stained with PTA 3% pH 6.0, and viewed using a Toshiba electron microscope. In these experiments, MV antigen vaccine formulations and control Proteosomes or OMP-LPS alone appear as round membrane structures of varying sizes, ranging in size from about 100 nm to about 300 nm (FIG. 5B). Immunogold® label signal clearly indicates the co-localization of MV antigen with Proteosomes.

### Example 9

### Murine Immunization with Measles Virus Antigen Vaccine Formulations

Immunizations were performed on 21 groups of 10 week old BALB/c female mice, with 5 mice per group. For each experiment, all mice were evaluated every 2 days for body weight and signs of toxicity (e.g., fur condition, hunched posture, oily skin, eye secretions and dehydration) throughout the course of the experiment. BALB/c mice were immunized intramuscularly (IM) and/or intranasally (IN) on day 1 and 14 with a Proteosome:MV antigen formulation, or a Proteosome:LPS:MV antigen formulation. All vaccine formulations contained 0.4 μg of MV antigen as prepared in Example 4. For IN immunizations, mice were first lightly anesthetized by inhalation of isofurane, and then presented with vaccine or control formulations using an automated induction chamber delivering 25 μl into the nares (12.5 μl per nostril). For IM immunization, 25 μl of vaccine formulation was presented by injection into the hind limb. In all cases, control mice were immunized IN or IM with buffer (PBS) alone, MV antigen alone, Proteosomes alone, or Proteosome:LPS (e.g., OMP:LPS) alone.

For analysis, samples were obtained from the lateral saphenous vein on day 1, on day 14, and, for mice receiving 3 doses of vaccine formulation, also on day 28. Eight days after the final immunization (day 22 and day 36 for the 2 and 3 dose experimental groups, respectively), mice were euthanized by asphyxiation with CO₂, and exsanguinated by cardiac puncture. Nasal and lung lavages were also performed by making an incision in the trachea and inserting a catheter (Clear-Cath®, Abott, Ireland), first into the major airways, and subsequently into the nasopharynx. For each location, the catheter was fixed by a suture and sampled with 1 ml PBS containing 0.1% BSA plus protease inhibitors (AEBSF, EDTA, bestatin, E-64, leupeptin and aprotinin (Sigma®, St. Louis, Mo.). All samples were collected and stored at −20° C. until used. Spleens were also collected from each mouse and splenocytes were prepared using 70 μm Nylon cell strainers (BD Falcon). Single cell suspensions were centrifuged using Ficoll®-Hypaque (Pharmaciag) at 280×g for 20 minutes. Cells located at the plasma/Ficoll® interface were collected, washed two times, and frozen in fetal calf serum containing 10% dimethyl sulfoxide (DMSO).

### Example 10

### Analysis of Antibodies Specific for Measles Virus by ELISA

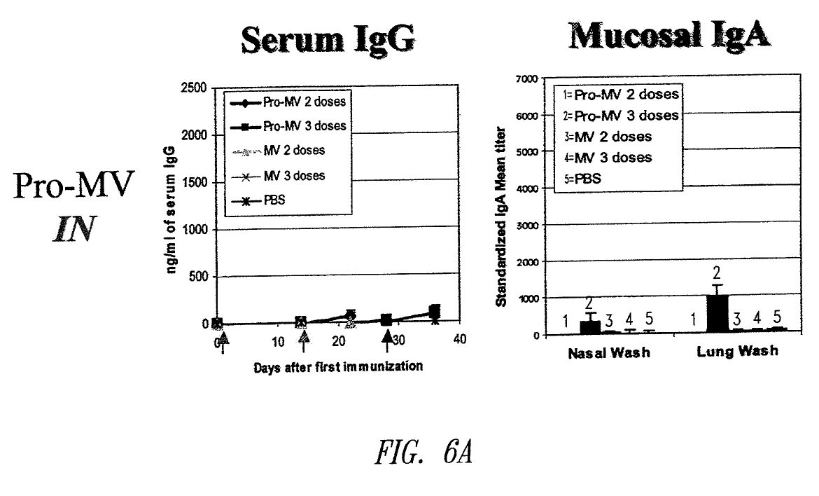

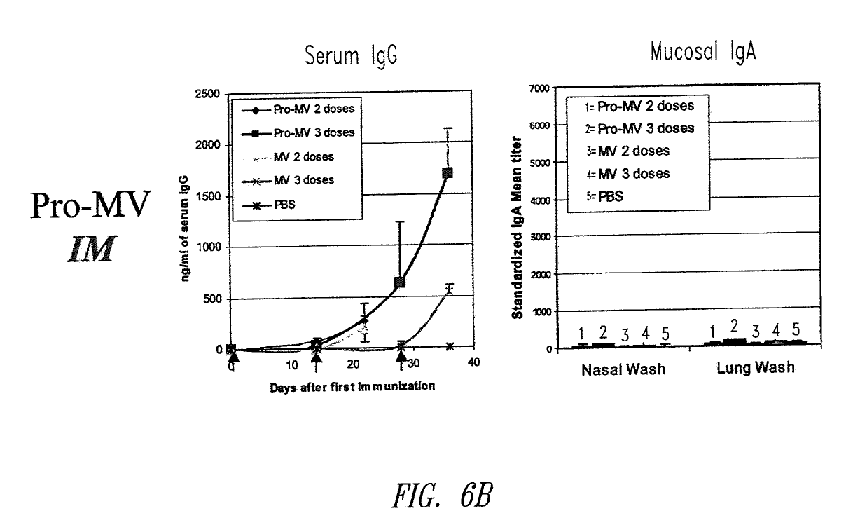

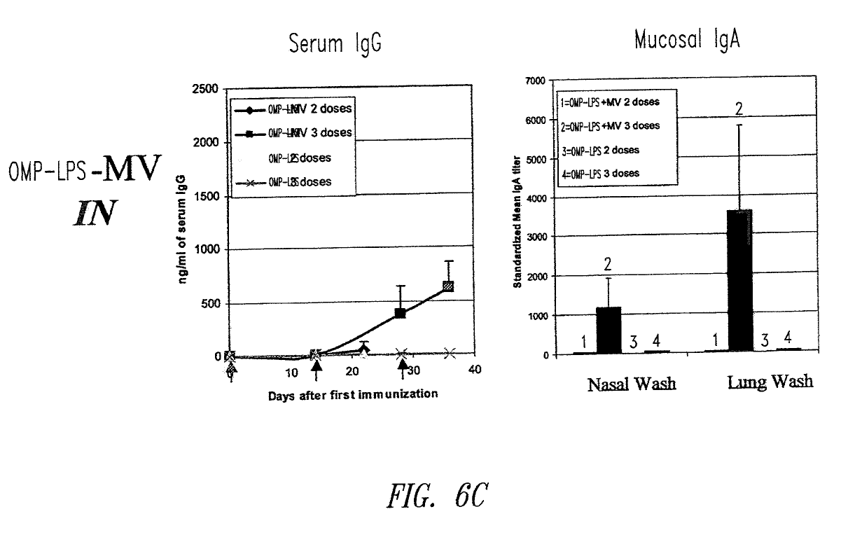

Serum and mucosal MV specific antibody responses were measured by quantitative ELISA. For serum samples, total IgG, IgG isotypes (IgG₁, IgG₂ₐ, IgG_{2b}) and IgA were measured. For nasal and lung washes, only IgA was assessed. U-bottom, 96-well microtiter plates (Greiner®) were coated overnight at 4° C. with 1 μg/ml My antigen diluted in carbonate buffer, pH 9.6. Plates were blocked with 2% skim milk in PBS 0.1% Tween-20 (PBS-T) before dilutions of samples were added in duplicate and incubated for a period of 2 hours at 37° C. Secondary antibodies include goat-anti-mouse IgG-horse radish peroxidase (HRP; Pharmingen® BD), goat anti-mouse IgG₁-HRP, goat anti-mouse IgG₂ₐ-HRP, or goat anti-mouse IgG_{2b}-HRP (Southern Biotechnologies Associates). A rat anti-mouse IgA-Biotin (Pharmingen®, BD) was used as a secondary antibody for the detection of IgA, followed by Streptavidin-HRP (Jackson Immunoresearch Laboratories). Assays were completed by the addition of TM Blue substrate (Serological Corporation). Reactions were stopped using 0.2 M sulfuric acid (Sigma®). The mean and standard deviation (SD) of optical density values recorded at 450 nm were calculated from an automatic microplate reader (Bio-Rad® Laboratories, Richmond, Calif.). The antibody concentrations in the test samples were calculated from a standard curve included on each plate using purified mouse IgG antibodies (Sigma®, St. Louis, Mo.) or purified mouse IgA (Bethyl Laboratories, Montgomery, Tex.). Values are expressed in nanograms of specific antibody per milliliter of serum or lavage fluid (see FIG. 6).

### Example 11

### Analysis of Antibodies Specific for Measles Virus by Plaque Reduction Neutralization Assay

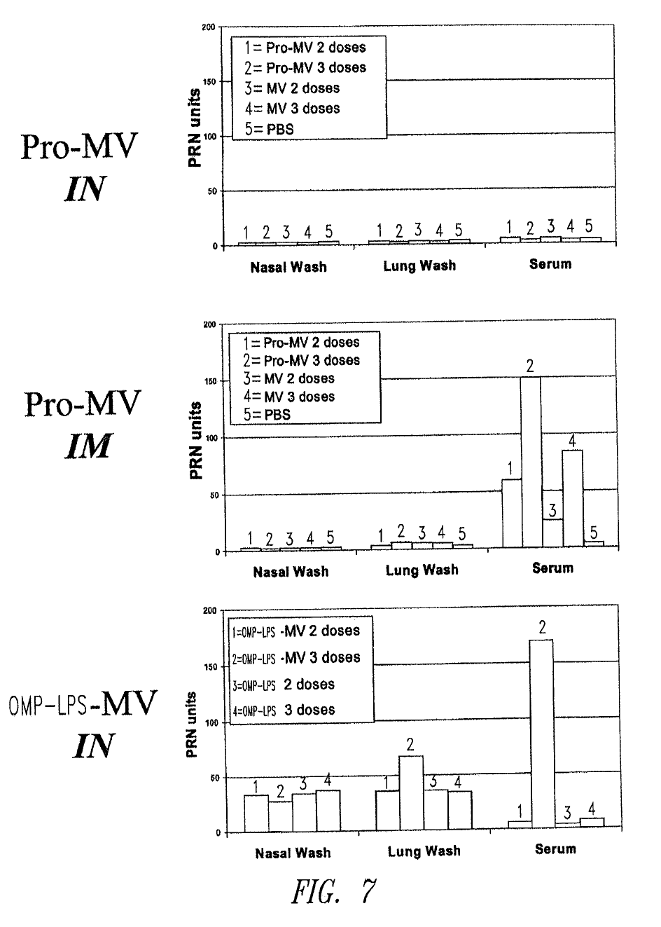

The ability of MV-specific antibodies contained in samples (serum and/or lavage) to neutralize the growth of MV was assessed by plaque reduction neutralization (PRN) assays as previously described (Ward et al., Diagn. Microbiol. Infect Dis. 33:147, 1999). Briefly, Vero cells were plated in 24-well plates (Falcon, BD Biosciences, Mississauga, Ontario, Canada) to obtain 90-95% confluence. Samples were heat-inactivated at 56° C. for 40 minutes before use in PRN. Samples were diluted and incubated with MV for a period of 90 minutes at 37° C. after which duplicate wells of 70% confluent Vero cells were infected with 100 μl of 10-fold serial dilutions. A 16% methylcellulose overlay in Lebovitz's L15 media (Gibco® Life Technologies, Grand Island, N.Y.) was applied to infected cells and plates were then incubated at 37° C. in 5% CO₂ for 4 days. A solution of 4% neutral red was added to stain the monolayer, and then left for an additional 24 hours. Finally, the cell monolayers were fixed with 3.7% formalin for 10 minutes and visible plaques were counted to determine the number of plaque forming units (see FIG. 7). Each sample was evaluated in duplicate. The PRN index was determined using the Kaber method to calculate the 50% end point of neutralization. The following formula was used to calculate the PRN value: log 10 of reciprocal of highest dilution−[(sum of the average plaque counts/average plaque count from virus control−0.5)×Log 10 of dilution factor].

### Example 12

### Safety of Measles Virus Antigen Vaccine Formulations

MV antigen vaccine formulations were evaluated for safety (i.e., toxicity) in mice (see also Example 9). Mice were immunized intranasally (IN) with either 2 or 3 doses of MV antigen formulated with Proteosomes or OMP-LPS. In addition, MV antigen formulated with Proteosomes were used to immunize mice via the intramuscular route (IM). No toxicity was detected using any of the vaccine formulations disclosed herein. The mice were observed and weighed every other day. For vaccine formulations administered either IN or IM, no behavioral changes were noted in any of the mice, and no statistically significant fluctuation in weight was detected (e.g., greater than +/−1.0 gram). These data suggest that the vaccine formulation of the instant invention would likely be safe for use with human subjects.

### Example 13

### Serum IgG Antibody Response Following Immunization With Measles Virus Antigen Vaccine Formulation

The ability of MV antigen vaccine formulations to elicit systemic immunity was assessed by analyzing serum samples by quantitative ELISA for MV-specific antibodies. In these experiments, mice were immunized on days 1, 14 and/or 28, as depicted in FIG. 6 (arrows). In these experiments, two or three doses of a Proteosome-MV antigen vaccine formulation administered IN induce a statistically significant increase in measurable amounts of IgG (FIG. 6). When administered IM, Proteosome-MV vaccine formulations elicited a measurable increase in serum IgG in all mice after receiving two doses of vaccine, which increased significantly after administering the third dose (FIG. 6). Detectable levels of serum IgG were also observed in mice receiving three doses of MV alone, administered IM. Mice immunized IN with OMP-LPS-MV antigen vaccine formulation developed significant levels of IgG following three doses of vaccine. Administration of two doses of OMP-LPS-MV antigen formulated vaccine did not elicit a detectable serum IgG response. Serum IgG was undetectable in all animals before immunization and remained undetectable at all time points in control groups, including groups receiving PBS control, MV antigen alone administered IN (2 or 3 doses) and OMP-LPS alone (2 or 3 doses) (FIG. 6).

### Example 14

### Mucosal Antibody Response Following Immunization with Measles virus antigen vaccine formulation

ELISA for the detection of IgA in nasal and lung ravages was used to determine the ability of MV vaccine formulations to elicit nasal and respiratory mucosal immunity. Nasal and lung lavages were performed 8 days after the last immunization. Significant levels of MV-specific IgA were detected in mice receiving 3 doses of Proteosome:MV vaccine formulations delivered IN, whereas such levels were not detected in mice receiving 2 doses of vaccine (FIG. 6). In contrast, 2 or 3 doses of Proteosome:MV formulated vaccine administered IM did not elicit a detectable IgA response (FIG. 6). Mice immunized with 3 doses of OMP-LPS-MV antigen formulated vaccine delivered IN elicited significant levels of MV-specific IgA with titers approaching 6000 ng/ml in lung lavage samples. IgA levels were higher in lung lavages than in nasal lavages (FIG. 6), suggesting that IN delivered OMP-LPS-MV antigen vaccine formulations elicit a mucosal immune response in the respiratory tract. OMP-LPS-MV antigen formulated vaccine delivered IM (2 doses) did not elicit a detectable IgA response. Levels of IgA remained low or undetectable in both nasal and lung lavages obtained from control groups, including groups receiving MV antigen alone delivered IN, MV antigen alone delivered IM (2 or 3 doses), PBS control and OMP-LPS alone delivered IN (2 or 3 doses).

### Example 15

### Immunoneutralizing Activity of Serum and Lavage Samples from Mice Immunized with a Measles Virus Antigen Vaccine Formulation

Plaque reduction neutralization assays were used to analyze serum samples, as well as nasal and lung lavage samples, for immunoneutralizing activity when collected 8 days after the last immunization. In these experiments, Proteosome-MV antigen formulated vaccine delivered IN showed low but significant MV neutralization (FIG. 7). Proteosome-MV vaccine formulations delivered IM did not elicit a mucosal IgA response (FIG. 6) and, consequently, no viral neutralization was observed when MV was exposed to nasal or lung lavage samples. Serum samples obtained from Proteosome-MV vaccine formulations delivered IM (3 doses) were shown to neutralize the growth of MV (FIG. 7). Significantly lower levels of neutralization were observed with serum samples from mice receiving Proteosome-MV antigen formulations delivered IM (2 doses), and from mice receiving MV antigen alone delivered IM (3 doses), as compared to Proteosome-MV antigen vaccines administered IM (3 doses) (FIG. 4), consistent with IgG levels measured by ELISA (FIG. 6). In addition, 3 doses of OMP-LPS-MV antigen vaccine formulations delivered IN elicited the production of neutralizing serum antibodies. Similar results were observed for Proteosome-MV formulations delivered IN. Detectable levels of immunoneutralizing activity were also observed in lung lavage samples (FIG. 7). Samples obtained from control groups such as MV antigen alone delivered IN (2 or 3 doses), PBS alone, and OMP-LPS alone (2 or 3 doses) had no MV neutralizing activity. These data suggest that MV antigen vaccine formulations elicit an immune response capable of protecting a vaccinated subject from MV infection and complications thereof.

### Example 16

### Immune Response in Mice Immunized with a Measles Virus Antigen Vaccine Formulation

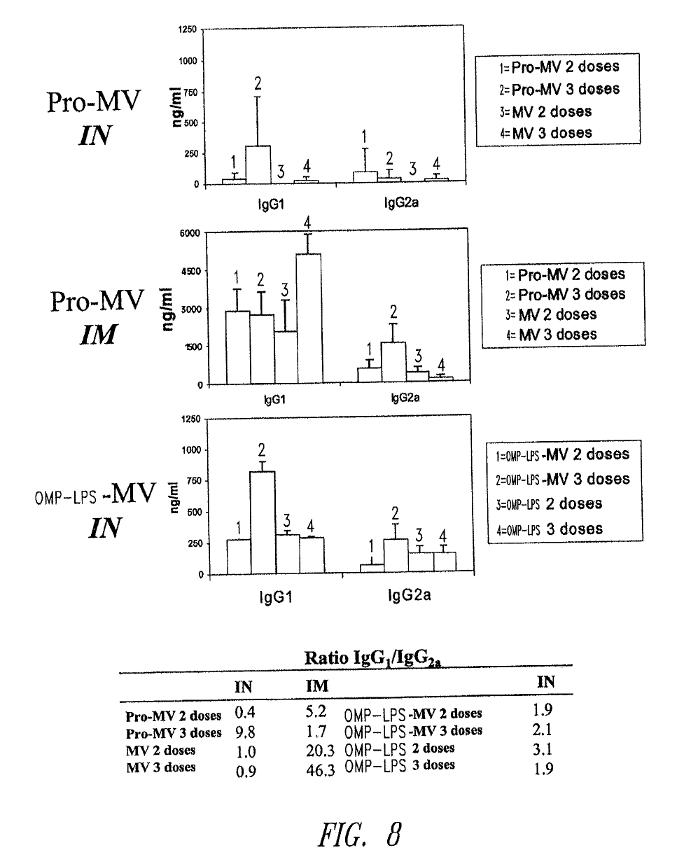

The predominance of different IgG antibody isotypes is associated with a specific type of immune response. Serum IgG₁ is associated with a T_{H}2-type response involved in humoral immunity, whereas serum IgG₂ₐ is associated with a T_{H}1-type response involved in cellular mediated immunity. The concentration of isotype-specific IgG₁ or IgG₂ₐ antibodies was measured by ELISA in serum collected 8 days after the last immunization. FIG. 8 shows IgG₁ and IgG₂ₐ levels in serum samples and respective IgG₁/IgG₂ₐ ratios for each experimental group. For groups where serum IgG responses were significantly higher than controls, formulation of MV with Proteosomes or OMP:LPS resulted in lower IgG₁/IgG₂ₐ ratios compared with MV alone groups administered IN or IM, which indicates that Proteosomes and OMP:LPS are efficient at redirecting the MV-specific immune response towards a type 1 phenotype.

### Example 17

### Analysis of Serum Antibody Specificity to Measles Virus Antigen

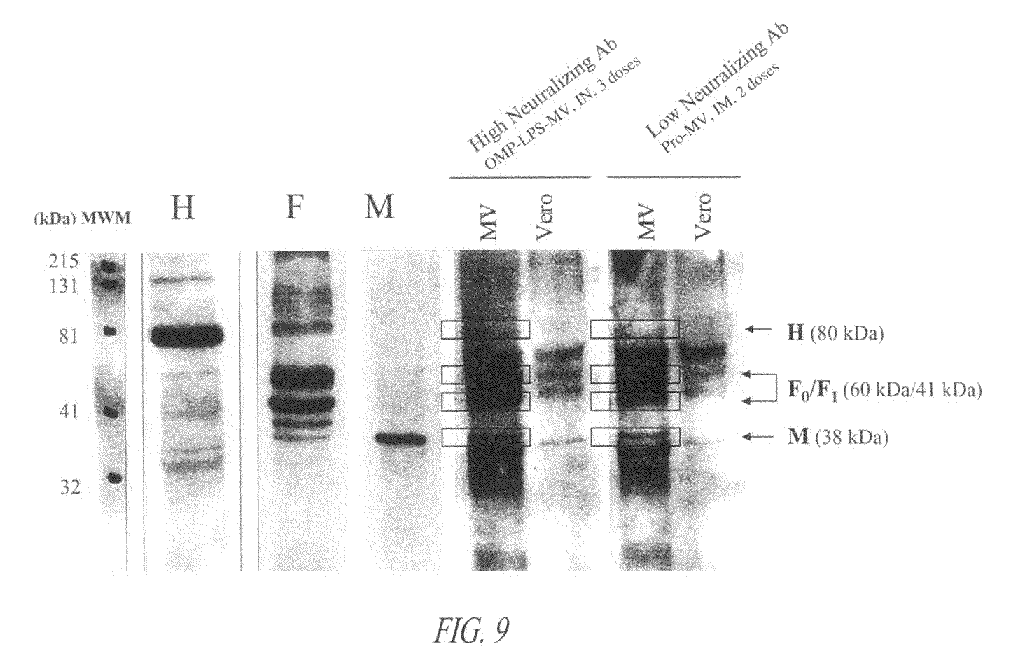

Antigen specificity of serum antibodies was determined by immunoblot analysis. Comparative immunoblots were designed to detect MV H, F, and M proteins (FIG. 9). Serum having MV neutralizing antibodies (obtained from mice immunized with OMP-LPS-MV antigen vaccine formulations delivered IN, 3 doses) was compared to serum having non-neutralizing antibodies (obtained from mice immunized with Proteosome-MV antigen vaccine formulations delivered IM, 2 doses). Serum containing antibody with high neutralizing activity was capable of specifically binding to MV proteins. In contrast, serum samples with low neutralizing activity did not detect MV H protein, and weakly recognized MV M protein. Recognition of MV F protein was difficult to detect over non-specific background binding because of the presence of cross-reactive Vero cell proteins of similar molecular size. Nevertheless, intensities of the F₀/F₁ bands was greater when immunoblots were exposed to neutralizing antibodies as compared to non-neutralizing antibodies. Taken together, these results demonstrate that the level of neutralizing activity correlates with the level of MV antigen recognition, for example, recognition of MV H protein.

### Example 18

### Immune Response of Animals Receiving Varying Doses of Measles Virus Antigen-Proteosome:LPS Formulation

This Example describes the mucosal and systemic neutralizing antibody immune response in mice that received varying doses of an intranasal measles virus vaccine formulated with Proteosome:LPS (IVX908).

### Measles Antigen Preparation

Measles virus split antigen was prepared as follows. Moraten vaccine-strain MV (gift from R. Wittes, Connaught Laboratory, Mississauga, ON) was grown in Vero green monkey kidney cells at a multiplicity of infection (MOI)=0.01-0.001 using 10 level cell factory chambers (Nalge® Nunc® International, Rochester, N.Y.). At peak cytopathic effect, flasks were freeze-thawed once. Cell debris was removed by centrifugation (2100×g for 20 min at 4° C.); pooled supernatants were filtered first through a 0.45 μm filter and then through a 0.22 μm filter. The filtrate was ultracentrifuged (10,000×g for 2 hours at 4° C.), and the pellet was resuspended and sonicated in phosphate-buffered saline (PBS) solution. Protein concentration was measured based on a standard curve using a mixture of bovine serum albumin fraction V and bovine gamma globulin fraction II (PierceE Bicinchoninic Acid Protein Assay, Pierce® Biotechnology, Rockford, Ill.). Priorto formulation with IVX908, 1% detergent (Mega-10 (n-Decanoyl-N-methylglucamide), Bachem AG) was added to the MV antigen preparation, which was then dialyzed against PBS for 7 days in a Slide-A-Lyzer dialysis cassette (Pierce®, Rockford, Ill.).

### Measles Virus Antigen Characterization

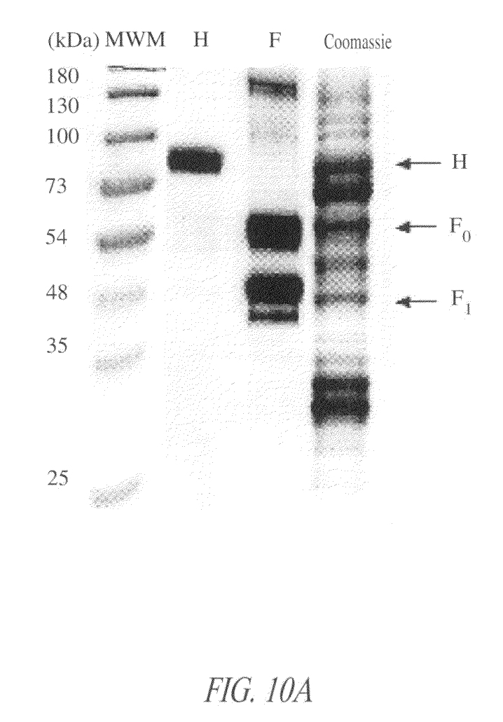

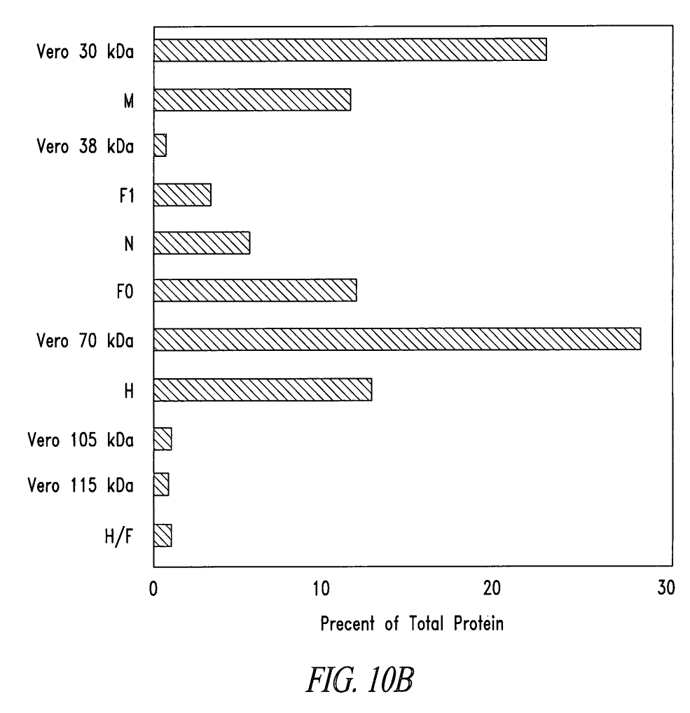

Serial dilutions of the MV split antigen were separated by electrophoresis on a 10% polyacrylamide gel, and protein bands were visualized with Coomassie® Blue G-250 (Kodak®, Rochester, N.Y.) (FIG. 10A). A band of 80 kDa corresponding to the MV H protein was observed. As expected, various F protein bands were obtained. F₀ was identified as a 60 kDa protein, and the proteolytically processed F₁ subunit was seen as a 41 kDa protein band. Other MV antigens identified by Coomassieg staining included N protein (50-60 kDa) and M protein (38 kDa). The presence of residual Vero proteins in the MV antigen preparation was also observed by Coomassie® staining, for example a dense band of about 70 kDa, which was detected in Vero cell lysate alone.

As shown in FIG. 10B, the relative and absolute amounts of individual MV proteins present in the antigen preparations were estimated by quantitative densitometric analysis of Coomassie®-stained gels using Scion Image software. The contribution of each band to the total protein was evaluated, and the proportion attributable to the H and F antigens was determined. The H and F protein accounted for ˜30% of total proteins in the MV preparations.

MV antigens run on a parallel gel were transferred to PVDF membranes for immunoblot analysis. Membranes were blocked with 5% skim milk-PBS containing 0.1% Tween-20 (PBS-T) before incubation for 1 hour at room temperature (RT) with monoclonal anti-F or anti-H antibodies (provided by Fabian Wild, Institut Pasteur de Lyon, France). Following washing with PBS-T, PVDF membranes were exposed to goat-anti-mouse-HRP (Jackson Immunoresearch Laboratories, West Grove, Pa.) for one hour at room temperature. Membranes were immersed in HRP substrate and binding of the goat anti-mouse HRP conjugate to the PDVF membranes was visualized using an ECL assay performed according to the manufacturer's instructions (ECL kit, Amersham Biosciences, Piscataway, N.J.). The results are presented in FIG. 10A.

### Preparation of Proteosome-Based Measles Vaccines (IVX908-MV)

IVX908 (also known as Protollin™) was manufactured under cGMP guidelines and was identical to the Proteosome-S. flexneri 2a LPS lot of the prospective S. flexneri vaccine prepared by diafiltration as previously described (Rima et al., Curr. Topics Microbiol. Immunol. 191:65-83 (1995)). The ratio of Proteosome porins to LPS is approximately 1:1 wt/wt. N. meningitidis Porin A, Porin B, and class IV protein constitute about 20%, 75%, and 5% of total Proteosome protein content, respectively. IVX908 was mixed with the MV antigen preparation in a 1:1 ratio immediately before administration to animals.

### Characterization of IVX908-MV by Electron Microscopy

Vaccine formulations were centrifuged in an airfuge onto nickel grids for 5 minutes. Grids were then immersed in a blocking solution of 1% BSA for 5 minutes. Monoclonal anti-H (Chemicon® International, Temecula, Calif.) was used as the primary antibody. Following one hour incubation at room temperature, grids were further blocked with 1% BSA for 5 minutes, and then exposed to anti-mouse IgG-Gold-10 nm (Aurion, Wageningen, Netherlands) for one hour at room temperature. Grids were washed with PBS and double distilled water before being air dried. Finally, the grids were colored with PTA 3% pH 6.0 (phosphotungsic acid) and viewed using a Hitachi® 7100 electron microscope. Representative electromicrographs are presented in FIG. 10C. IVX908 appeared as round membrane structures of varying sizes (100 nm to 300 nm) (FIG. 10C). The close association of gold particles with the surface of the IVX908 structures indicated that MV antigens were associated with IVX908 in the vaccine formulation. H antigen that was not associated with IVX908 was also observed.

### Animal Study Procedures and Sample Collection

All animal procedures were approved by McGill University Animal Care and Use Committee (Protocol #4481). Twenty-one groups of BALB/c 10-week old female mice were used (5 animals per group). Body weight and signs oftoxicity (i.e., fur erection, hunched posture, oily skin, eye secretions, dehydration) were monitored every 2 days throughout the experiment. Table 1 describes the different experimental groups studied. Vaccine formulations contained MV antigens at concentrations of 1, 3, and 6 μg per dose, whereas the concentration of IVX908 remained constant at 3 μg/dose for all formulations. PBS was used as a diluent for all vaccine formulations. Control groups received IVX908 alone (3 μg/dose), MV antigen alone (1, 3, or 6 μg/dose), Vero cell protein alone (6 μg/dose), IVX908-Vero cell protein (6 μg/dose), and vehicle PBS.

All vaccines were administered intranasally (IN). Immunizations were performed under isoflurane anesthesia using an automated induction chamber, and 25 μl of vaccine were instilled into the nares (12.5 μl per nostril) using a pipet gun and sterilized tips. Mice were immunized every 2 weeks on days 1 and 14, and on day 28 for groups receiving a third dose. Mice were bled from the lateral sapheneous vein before every immunization and ten days after the last immunization (day 24 and day 38 for 2 and 3 doses groups, respectively). All doses of IVX908-MV were well tolerated for both primary and booster immunizations. No behavioral changes were noted, and very little fluctuation in weight (±1.0 gram) was observed. Small numbers of animals (<10%) that were immunized intranasally with IVX908 alone or with IVX908-MV had oily fur and hunched posture for up to 5 days following immunization. However, no weight loss was observed in those mice.

On terminal days, mice were sacrificed by asphyxiation with CO₂, after which they were exsanguinated by cardiac puncture. Nasal and lung lavages were preformed by making an incision in the trachea and inserting a 12 G-catheter (Clear-Cath®, Abbott, Ireland), first into the major airways and subsequently into the nasopharynx. For each position, the catheter was fixed by a suture and 1 ml of PBS containing 0.1% BSA and a protease inhibitor cocktail (containing a mixture of AEBSF, EDTA, bestatin, E-64, leupeptin, and aprotinin (Sigma®, St-Louis, Mich.)). Wash fluid was collected by aspiration of the lung lavage or by catching drops from the nostrils. All fluids were stored at −20° C. until used. On the terminal day, spleens were aseptically removed and a single-cell suspension was prepared using 70 μm Nylon cell strainers (BD Falcon). Splenocytes were resuspended in RPMI 1640 (Wisent) supplemented with 10% fetal bovine serum (GIBCO) and 1 ug/ml gentamicin (Wisent).

TABLE 1
DESCRIPTION OF EXPERIMENTAL GROUPS

| | Dose of MV | Dose | | No. | No. |
|---|---|---|---|---|---|
| | Split Antigen | IVX908 | | of | of |
| Animal Group | (μg) | (μg) | Route | animals | doses |
| MV split antigen 1 μg | MV 1 | N/A | IN | 5 | 3 |
| MV split antigen 3 μg | MV 3 | N/A | IN | 5 | 3 |
| MV split antigen 6 μg | MV 6 | N/A | IN | 5 | 3 |
| IVX908 + MV 1 μg 3X | MV 1 | 3 | IN | 5 | 3 |
| IVX908 + MV 3 μg 3X | MV 3 | 3 | IN | 5 | 3 |
| IVX908 + MV 6 μg 3X | MV 6 | 3 | IN | 5 | 3 |
| IVX908 + MV 1 μg 2X | MV 1 | 3 | IN | 5 | 2 |
| IVX908 + MV 3 μg 2X | MV 3 | 3 | IN | 5 | 2 |
| IVX908 + MV 6 μg 2X | MV 6 | 3 | IN | 5 | 2 |
| IVX908 3X | N/A | 3 | IN | 5 | 3 |
| IVX908 2X | N/A | 3 | IN | 5 | 2 |
| Vero prep 6 μg 3X | Vero 6 | N/A | IN | 5 | 3 |
| IVX908 − Vero 6 μg 3X | Vero 6 | 3 | IN | 5 | 3 |
| PBS 3X | N/A | 3 | IN | 5 | 3 |
| Non-manipulated | N/A | N/A | N/A | 5 | N/A |

Quantification of MV-Specific Antibodies by ELISA Serum and mucosal MV-specific antibody responses were measured by quantitative ELISA. In sera, total IgG and specific IgG isotypes (IgG₁, IgG₂ₐ) were measured. In nasal and lung washes, MV-specific IgA levels were determined. Round-bottom 96-well microtiter plates (Greiner®, MJS Biolynx, Brockville, ON) were coated overnight at 4° C. with 1 μg/ml of whole sonicated MV antigen diluted in carbonate-bicarbonate buffer (pH 9.6). Plates were blocked with 2% skim milk-PBS-T before dilutions of samples in duplicate were added and incubated for 2 hours at 37° C. After washing with PBS-T, secondary labeled antibodies were added for 1 hour at 37° C. Secondary antibodies included goat-anti-mouse-IgG-HRP (Pharmingen® BD, San Diego, Calif.), goat-anti-mouse-IgG₁-HRP, goat-anti-mouse-IgG₂ₐ-HRP, and goat-anti-mouse-IgG_{2b}-HRP (Southern Biotechnologies Associates, Birmingham, Ala.). For IgA detection, rat-anti-mouse IgA-Biotin (Pharmingen®, BD, San Diego, Calif.) was used as a secondary antibody followed by streptavidin-HRP (Jackson Immunoresearch Laboratories, West Grove, Pa.). Assays were completed by the addition of TM Blue substrate (Serologicals Corporation, Norcross, Ga.). Reactions were stopped using 0.2 M sulfuric acid (Sigma-Aldrich® Canada, Oakville, Ontario). Serial dilutions of each sample were measured, and optical density values of the data points falling within the 25%-75% range of the standard curve were chosen to generate the final estimated concentration. The means and standard deviations (S.D.) of optical density values at 450 nm were calculated from an automatic microplate reader (Bio-Rad® Laboratories, Richmond, Calif.). Antibody concentrations in the test samples were calculated from standard curves run on each plate using purified mouse IgG (Sigma-Aldrich® Canada, Oakville, Ontario) or purified mouse IgA (Bethyl Laboratories, Montgomery, Tex.). Values are expressed in ng/ml of specific antibody in serum or in lung/nasal lavage fluid. Seroconversion after immunization with Proteosome-based vaccine was defined as at least a 4-fold rise in antibody titer from pre-vaccination levels.

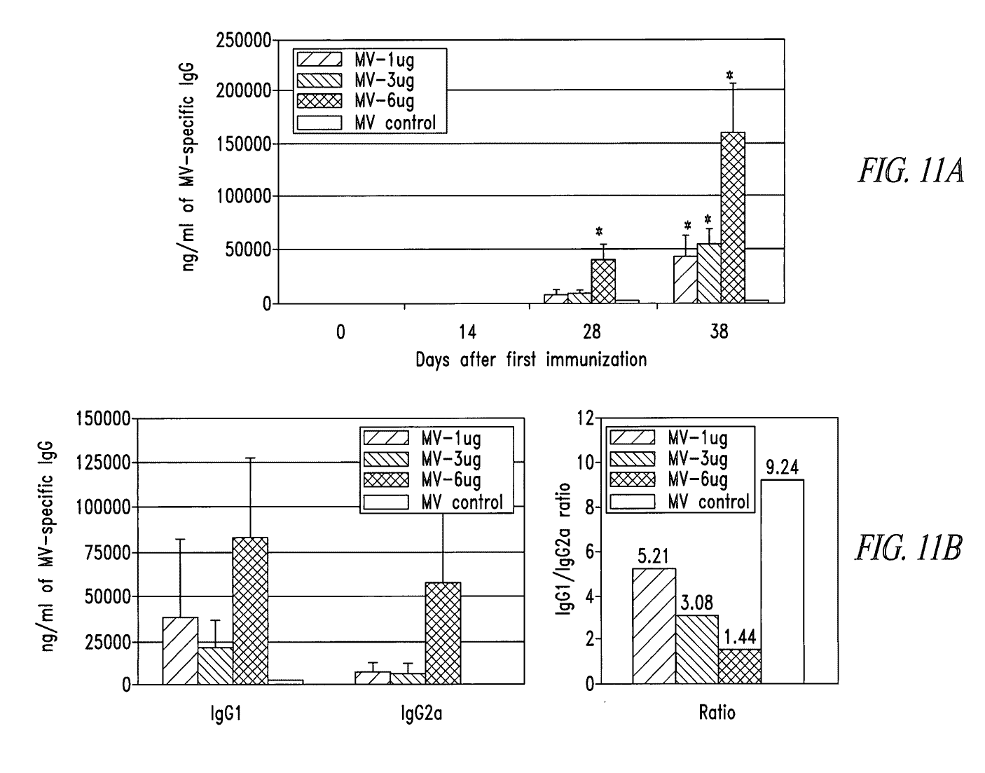

Data presenting total MV-specific IgG present in serum of Balb/c mice that was measured by ELISA on immunization days (1, 14, 28) and terminal days (24 or 38) is provided in FIG. 11. Values are expressed as mean IgG concentration+/−SEM. Serum antibodies were undetectable in most animals in all study groups after the first dose of vaccine. Animals immunized with IVX908-MV seroconverted after the second immunization (FIG. 11A), suggesting that at least one booster dose was beneficial for eliciting an immune response. Significantly higher levels of MV-specific serum IgG were achieved in animals that received a third immunization. The increases in serum IgG levels were dependent on the concentration of MV split antigen used for vaccine formulation. The correlation coefficient (R²) between MV antigen dose and serum IgG was 0.938 after 2 doses and 0.934 after 3 doses. MV alone (6 μg, MV control shown in FIG. 11A), IVX908 alone, 6 μg Vero protein, IVX-Vero, and PBS had undetectable or very low levels of IgG (values ranging from 1-500 ng/ml) (p<0.05 by one way analysis of variance (ANOVA) analysis, Bonferroni multiple comparisons test).

ELISA results for specific isotype antibodies were performed on serum samples of the terminal bleed of animals that received 3 doses of IVX-908-MV (FIG. 11B). On the left panel of FIG. 11B, values represent the mean concentration+/−SD of 5 animals. The ratio of IgG₁/IgG₂ₐ mean levels were calculated, and values are shown and plotted in the graph in the right panel of FIG. 11B.

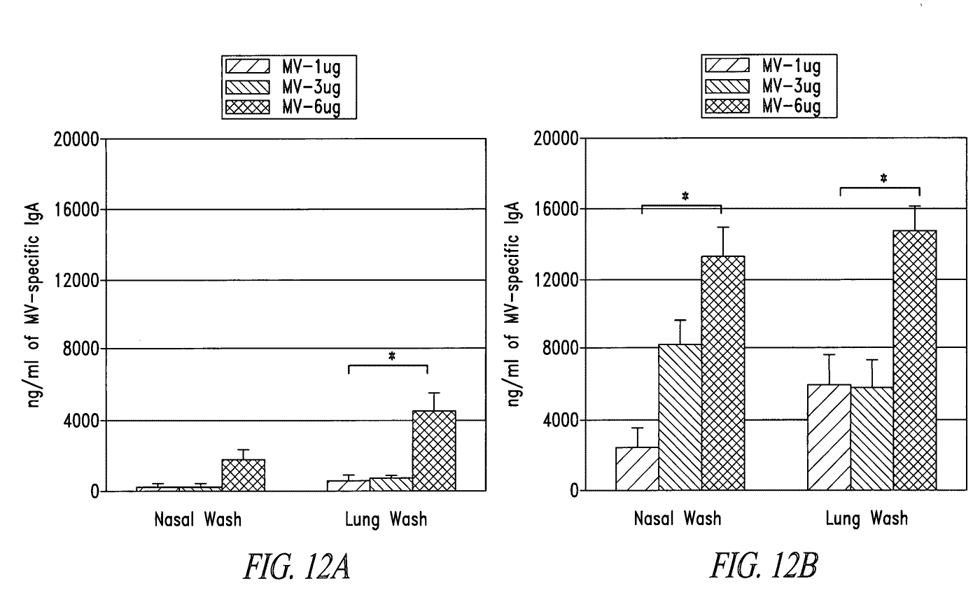

FIG. 12 presents ELISA data illustrating the level of IgA in nasal and lung washes obtained from animals 10 days after the last immunization (day 24 or day 38, for two dose or three dose immunization, respectively). After 2 doses, MV-specific IgA seroconversion was observed only in animals immunized with IVX908-MV containing 6 μg of MV split antigen, suggesting that the induction of an MV-specific mucosal response was antigen dose-dependent. Dose-dependency was also observed in the animals that received 3 doses. For animals that received 3 doses, the correlation coefficients between MV dose and IgA levels in nasal and lung lavages were 0.977 and 0.826, respectively. MV-specific IgA levels were similar in both lung and nasal fluid, suggesting that Protollin™-MV elicited mucosal responses in both the lower and upper respiratory tracts. Levels of IgA remained low or undetectable in respiratory mucosal secretions of all control groups.

### Plague Reduction Neutralization (PRN) Assays

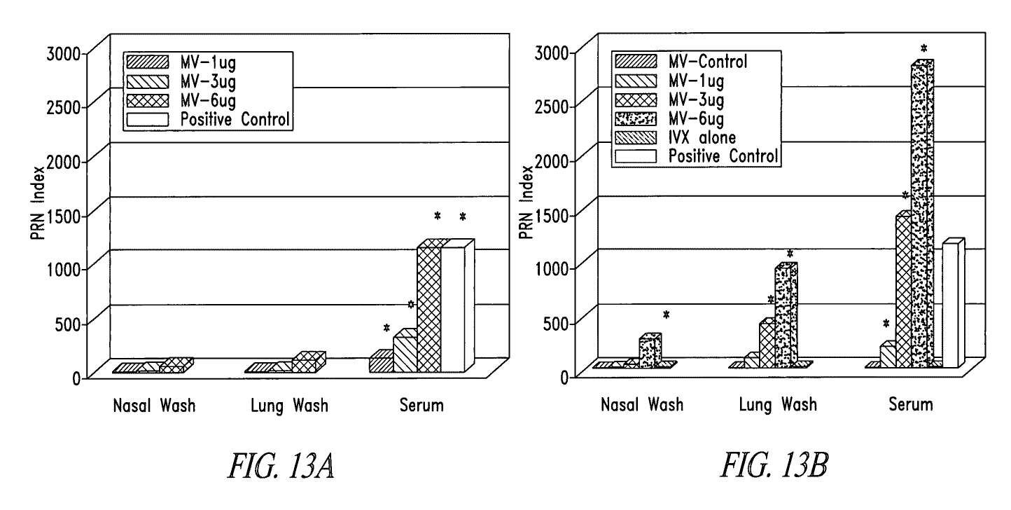

MV neutralizing antibodies were assessed by plaque reduction neutralization (PRN) assays as previously described (Ward et al., Diagn. Microbiol. Infect. Dis. 33:147-52 (1999)). Briefly, Vero cells were seeded in 24-well plates (Falcon, BD Biosciences, Mississauga, ON, Canada) to obtain 90-95% confluency. Serum samples were pooled from five animals in each experimental group and heat-inactivated at 56° C. for 40 minutes before use. Serial dilutions of sera were mixed and incubated with low-passage Edmonston MV (25-35 plaque-forming units) for a period of 90 minutes at 37° C. Duplicate wells of confluent Vero cells were then infected with 100 μl of 2-fold serial dilutions of the sera plus MV mixture. A 16% methylcellulose overlay in Liebovitz's L-15 media (Gibco®/Life Technologies, Grand Island, N.Y.) was applied to infected cells, and the cells were then incubated at 37° C. in 5% CO₂ for 4 days. A solution of 4% neutral red was added to stain the monolayers, and cells were incubated for an additional 24 hours. Cell monolayers were then fixed with 3.7% formalin for 10 minutes. Visible plaques were counted to determine the number of plaque forming units (PFU). Virus alone served as negative control, and human serum from an individual vaccinated with a measles virus vaccine served as a positive control. The PRN value was obtained using the Kaber method to determine the 50% end-point of neutralization. PRN values are expressed as the log₂ of the reciprocal of serum dilution that reduced the number of plaques by ≧50%. By way of comparison, seroprotection in humans has been defined as a PRN value>120 (Chen et al., J. Infect. Dis. 162:1036-42 (1990)). PRN values were standardized to antibody concentration. A graphic representation of neutralization activity of serum samples obtained from animals after receiving two doses and three doses of IVX908:MV antigen vaccine is presented in FIG. 13A and FIG. 13B, respectively. At all MV split antigen concentrations, two doses of IVX908-MV were sufficient to elicit a significant serum neutralizing activity. An additional dose of IVX908-MV enhanced the serum neutralizing response. Significant neutralization by antibodies present in nasal and lung fluids was also observed in the group receiving the highest MV split antigen concentration (6 μg) and in lung lavage fluids at 3 μg/dose. Serum and mucosal samples from control groups had no neutralizing activity at any time point.

### Detection of Anti-H Protein Antibodies in Sera

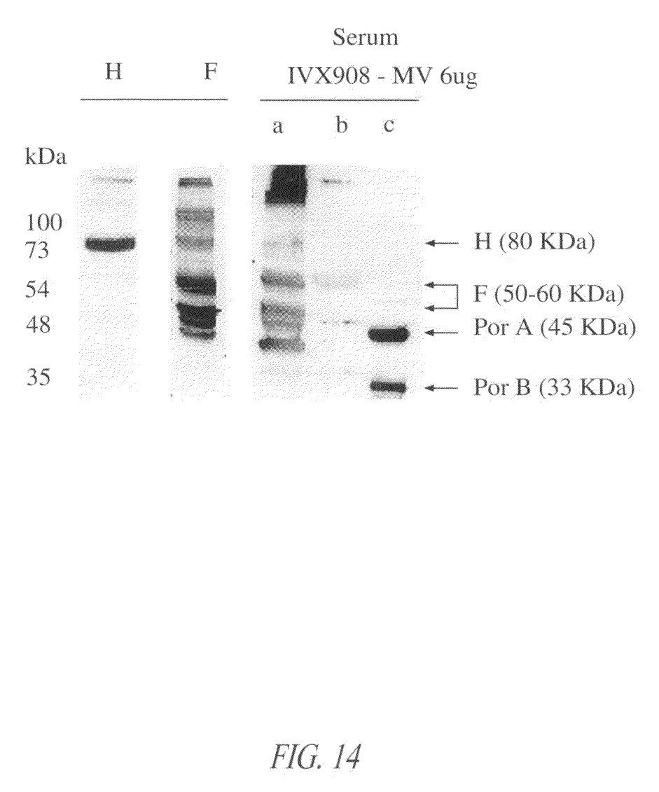

Sera were collected from animals that received 3 doses of IVX908-MV at 6 μg per dose with high neutralizing activity and were analyzed by immunoblot to detect antibodies specific for MV antigens. Immunoblot analyses of an MV split antigen (see method of preparation above) using monoclonal antibodies that specifically bind H and F MV antigens were also performed. Preparations of MV split antigen, Vero protein, and OMP Proteosome were separated by SDS-PAGE, and an immunoblot of the separated antigens was performed as described above. As shown in FIG. 14, serum collected from IVX908-MV-immunized mice recognized measles virus H protein (80 kDa); measles virus F₀ protein (50-60 kDa); measles virus F, protein (41 kDa); N. meningitidis OMP Por A (45 kDa); and N. meningitidis OMP Por B (33 kDa).

### Cytokine Detection by ELISPOT

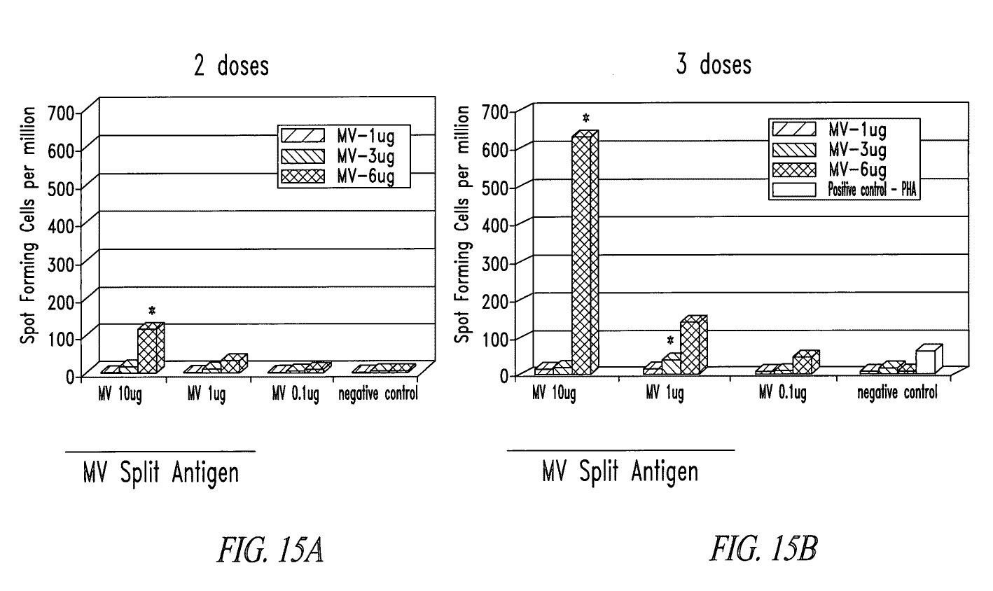

In mice, serum IgG₁ is associated with a T_{H}2-type response, whereas serum IgG₂ₐ is associated with a T_{H}1-type response (Maassen et al., Vaccine 21:2751-57 (2003)). Ten days after the last dose of vaccine, spleens were obtained from all mice. Mononuclear cells were isolated from the spleens by processing through a 70 μm Nylon cell strainer (BD Falcon) to obtain single cell suspensions. Splenocytes from five animals per experimental group (see Table 1) were pooled. MV-specific stimulation of IFNγ secretion by splenocytes was quantified by ELISPOT (Enzyme Linked ImmunoSPOT) (MABTECH, Nacka, Sweden). Splenocytes were seeded at a density of 100,000 cells/well in MultiScreen™ Immunobilon-P-based 96-well plaies (Millipore®, Billerica, Mass.) that were coated with 5 μg/ml of anti-IFNγ monoclonal antibody clone AN-18 diluted in carbonate/bicarbonate buffer (pH 9.6). Splenocytes were stimulated with different concentrations of MV split antigen (0.1 to 10 μg) for a period of 72 hours. PHA (5 μg/mL) was used as a positive control. Vero protein preparation (10 μg/mL) and culture medium were used as negative controls. Results are expressed as spot-forming cells (SFCs)/million splenocytes after subtracting negative control values. Negative controls produced less than 5 spots per well in most experiments (mean=1.3±1.2). Experimental wells were considered positive if more than 5 spots/well were present (>3 SDs above the mean). The mean number of spots induced by the Vero protein preparation alone (negative control values) was subtracted from the mean number of spots induced by different concentrations of MV split antigen, which was normalized to numbers of cytokine spot-forming T-cell subset per 100,000 cells (FIG. 15). Incubation of MV split antigen with splenocytes from control groups (IVX alone, MV alone, PBS) did not result in spot formation. These data indicate that IVX908-MV administered intranasally has the capability to induce an MV-specific IFNγ response. Values represent the mean of triplicate experiments (*p<0.05 T-test one tail of unequal variances).

Statistical analyses for the experiments in this Example were performed using Instat (GraphPad Software, San Diego, Calif.). Means obtained for the different test groups were compared using the Bonferroni multiple comparison ANOVA. In all tests, a p-value<0.05 was considered to be statistically significant.

### Example 19

### Immune Response in Monkeys Primed with a DNA Vaccine and Boosted with IVX908

This Example describes the immune response in juvenile rhesus macaque monkeys immunized with measles DNA vaccine followed by intranasal boosting immunization with IVX908. All animals in this study were cared for and treated in accordance with procedures and protocols for proper care of research animals.

Groups of juvenile rhesus macaques (measles seronegative) received two priming immunizations with a DNA vaccine construct, followed by a boosting immunization with either an attenuated measles vaccine or IVX908-MV. The DNA vaccine constructs included a plasmid comprising DNA that encoded MV H protein (PMSINH) and a bicistronic plasmid comprising DNA that encoded MV H protein and F protein (pMSINH-FdU). The plasmids were prepared according to methods known in the art. A third DNA vaccine, CVD 1208 (pMSIN/HF), was prepared by transfecting Shigella flexneri 2a strain CVD 1208 with a plasmid that encodes H protein and F protein, according to procedures similar to methods described in Pasetti et al. (J. Virol. 77:5209-17 (2003)). Animals received priming immunizations with a DNA vaccine twice, at day 0 and at Day 28. Each priming dose of pMSINH and pMSINH-FdU was 1 mg total, administered intradermally (i.d.) in 500 μg aliquots to two different legs using Biojector® (Bioject® Medical Technologies, Inc., Bedminster, N.J.). CVD 1208 (pMSIN/HF) bacteria were delivered intranasally (i.n.). At Day 59, animals were boosted with either an attenuated measles vaccine (e.g., Schwarz strain or Edmonston strain (ATCC, Manassas, Va.) that is attenuated according to standard protocols), delivered by aerosol according to methods known in the art or with IVX908-MV administered intranasally (i.n.) (50 μg total, 25 μg per nostril). The boosting immunizations were administered on Day 59. Controls included (1) 2 priming immunizations with PBS followed by a boosting immunization with aerosol delivery of the attenuated measles vaccine; (2) 2 priming immunizations with PBS followed by a boosting immunization with IVX908. An outline of the immunization protocol is presented in Table 2.

TABLE 2
ANIMAL GROUPS AND IMMUNIZATION SCHEDULE

| # Animals | Priming Immunization | | |
|---|---|---|---|
| per Group | Day 0 | Day 28 | Boost (Day 59) |
| 3 | pMSINH-FdU | pMSINH-FdU | Attenuated measles |
| | (i.d.) | (i.d.) | vaccine (aerosol) |
| 3 | pMSINH-FdU | pMSINH-FdU | IVX908-MV (i.n.) |
| | (i.d.) | (i.d.) | |
| 3 | CVD 1208 | CVD 1208 | Attenuated measles |
| | (pMSIN/HF) | (pMSIN/H-F) | vaccine (aerosol) |
| | (i.n.) | (i.n.) | |
| 2 | PBS (i.d.) | PBS (i.d.) | IVX908-MV (i.n.) |
| 2 | PBS (i.d.) | PBS (i.d.) | Attenuated measles |
| | | | vaccine (aerosol) |
| 3 | pMSINH (i.d.) | pMSINH (i.d.) | Attenuated measles |
| | | | vaccine (aerosol) |
| 2 | pMSINH (i.d.) | pMSINH (i.d.) | IVX908-MV (i.n.) |

Serum samples were obtained from the monkeys prior to priming immunizations at Day −7 and at Day 0 (pre-bleeds). Sera were then collected every few days, weekly, or biweekly after the animals received the first priming immunization.

The presence of MV antigen specific IgG antibodies in sera was determined by ELISA, which was performed according to standard procedures known to those skilled in the art. MV lysate ((Advanced Biotechnology, Colmbia, Md.). Table 3 presents the fold-increase in anti-MV antigen titer from Day 0 to Day 73 and to Day 91.

TABLE 3
MEASLES ANTIGEN SPECIFIC IGG RESPONSE

| | | | | Boost | | | |
|---|---|---|---|---|---|---|---|
| | Priming | | | Aerosol MV | | IVX908-MV | |
| Groups | Immunization | Route | Day 28 | Day 73 | Day 91 | Day 73 | Day 91 |
| 1A (n = 3) | pMSIN-H | i.d. | 34 | 223 | 193 | | |
| 1B (n = 2) | | | | | | 224 | 134 |
| 2A (n = 3) | pMSINH-FdU | i.d. | 7 | 117 | 181 | | |
| 2B (n = 3) | | | | | | 131 | 53 |
| 3 (n = 3) | CVD 1208 | i.n. | 1 | 15 | 136 | NA | NA |
| | (pMSINH/F) | | | | | | |
| 3A (n = 2) | PBS | i.d. | 1 | 1 | 34 | | |
| 3B (n = 2) | | | | | | 1 | 1 |

Measles virus antigen-specific IFNγ was determined using peripheral blood mononuclear cells (PBMCS) isolated from the animals according to methods known in the art for fractionating blood. PBMCs were stimulated with MV lysate (Advanced Biotechnology, Colmbia, Md.) (5 μg/ml) in nitrocellulose plates that were previously coated with IFNγ antibodies (Mabtech®). Results expressed as the mean number of spot forming cells per 10⁶ PBMCs are presented in Table 4.

TABLE 4
MEASLES VIRUS-SPECIFIC IFNΓ RESPONSE
IN JUVENILE RHESUS MACAQUES

| | | | | | Boost | | | |
|---|---|---|---|---|---|---|---|---|
| | Priming | | | | Aerosol MV | | IVX908-MV | |
| Groups | Immunization | Route | Day 0 | Day 59 | Day 73 | Day 91 | Day 73 | Day 91 |
| 1A (n = 3) | pMSIN-H | i.d. | 1 | 114 | 134 | 77 | | |
| 1B (n = 2) | | | | | | | 42 | 27 |
| 2A (n = 3) | pMSINH-FdU | i.d. | 2 | 65 | 228 | 36 | | |
| 2B (n = 3) | | | | | | | 102 | 35 |
| 3 (n = 3) | CVD 1208 | i.n. | 1 | 7 | 249 | 93 | NA | |
| | (pMSINH-FdU) | | | | | | | |
| 3A (n = 2) | PBS | i.d. | 1 | 10 | ND | 46 | | |
| 3B (n = 2) | | | | | | | 60 | 20 |

Sera collected from animals were analyzed for neutralizing activity in a PRN assay, performed according to methods known in the art and described in Example 18. The results for individual monkeys are presented in Table 5. By way of comparison, seroprotection in humans has been defined as a PRN value>120 (Chen et al., J. Infect. Dis. 162:1036-42 (1990)).

Seven animals received IVX908-MV and none of the animals exhibited any symptoms that indicated that the IVX908-MV formulation had any toxic or effected any adverse reaction in the animals. Thus, the IVX908-MV measles vaccine was safely administered to animals and induced a specific virus-neutralizing immune response.

The capability of the IVX908-MV measles vaccine to prevent animals from manifesting clinical symptoms of a measles infection is determined by challenging the monkeys in the groups as outlined in Table 2 with a strain of measles virus approximately one year after the boosting immunization. The animals are monitored for symptomatology indicating a measles infection and virus load is determined. The humoral immune response, both systemic and mucosal, is determined by methods described herein for measuring immunoglobulin levels in sera and nasal and lung ravages. The cell-mediated response induced in the animals is determined by methods known in the art and described herein.

TABLE 5
MV NEUTRALIZING ANTIBODIES IN SERA FROM IMMUNIZED MACAQUES

| | | | First dose | | | | | Second dose | | | Boost |
|---|---|---|---|---|---|---|---|---|---|---|---|
| | | Day | | | | | | | | | |
| ID# | Priming Immunization + Boost | −25 | 0 | 7 | 10 | 14 | 21 | 28 | 43 | 49 | 59 |
| 01R 0044 | pMSINH-FdU + Aerosol MV | 6.25 | 6.25 | 6.25 | 6.25 | 6.25 | 6.25 | 6.25 | 42.76 | — | 37.82 |
| 02R 0158 | pMSINH-FdU + Aerosol MV | 6.25 | 6.25 | 6.25 | 6.25 | 6.25 | 6.25 | 6.25 | 31.07 | — | 39.60 |
| 01R 2342 | pMSINH-FdU + Aerosol MV | 6.25 | 6.25 | 6.25 | 6.25 | 6.25 | 6.25 | 6.25 | >2,560 | >2,560 | >2,560 |
| 02R 0002 | pMSINH-FdU + IVX908-MV | 6.25 | 6.25 | 6.25 | 6.25 | 6.25 | 6.25 | 24.92 | 86.84 | 75.64 | 47.86 |
| 7Q | pMSINH-FdU ++ IVX908-MV | 6.25 | 6.25 | 6.25 | 6.25 | 6.25 | 6.25 | 6.25 | 6.25 | 6.25 | 59.41 |
| 42Q | pMSINH-FdU ++ IVX908-MV | 6.25 | 6.25 | 6.25 | 6.25 | 6.25 | 6.25 | 6.25 | 6.25 | 6.25 | 6.25 |
| 16Q | CVD1208(pMSIN/HF) + Aerosol MV | 6.25 | 6.25 | 6.25 | 6.25 | 6.25 | 6.25 | 6.25 | 6.25 | 6.25 | 6.25 |
| 11Q | CVD1208(pMSIN/HF) + Aerosol MV | 6.25 | 6.25 | 6.25 | 6.25 | 6.25 | 6.25 | 6.25 | 6.25 | 6.25 | 6.25 |
| 01R 2488 | CVD1208(pMSIN/HF) + Aerosol MV | 6.25 | 6.25 | 6.25 | 6.25 | 6.25 | 6.25 | 6.25 | 6.25 | 6.25 | 6.25 |
| 5Q | PBS + IVX908-MV | 6.25 | 6.25 | 6.25 | 6.25 | 6.25 | 6.25 | 6.25 | 6.25 | 6.25 | 6.25 |
| 25P | PBS + IVX908-MV | 6.25 | 6.25 | 6.25 | 6.25 | 6.25 | 6.25 | 6.25 | 6.25 | 6.25 | 6.25 |
| 01R 0894 | PBS + Aerosol MV | 6.25 | 6.25 | 6.25 | 6.25 | 6.25 | 6.25 | 6.25 | 6.25 | 6.25 | 6.25 |
| 01R 0650 | PBS + Aerosol MV | 6.25 | 6.25 | 6.25 | 6.25 | 6.25 | 6.25 | 6.25 | 6.25 | 6.25 | 6.25 |
| 29Q | pMSINH + Aerosol MV | 6.25 | 6.25 | 6.25 | 6.25 | 6.25 | 6.25 | 6.25 | >2,560 | >5,120 | >5,120 |
| 01R 0072 | pMSINH + Aerosol MV | 6.25 | 6.25 | 6.25 | 6.25 | 6.25 | 6.25 | 6.25 | >2,560 | >5,120 | >5,120 |
| 01R 0742 | pMSINH + Aerosol MV | 6.25 | 6.25 | 6.25 | 6.25 | 6.25 | 6.25 | 6.25 | >2,560 | >5,120 | >5,120 |
| 24Q | pMSINH + IVX908-MV | 6.25 | 6.25 | 6.25 | 6.25 | 6.25 | 6.25 | 6.25 | 589.69 | 974.33 | 950.62 |
| 10Q | pMSINH + IVX908-MV | 6.25 | 6.25 | 6.25 | 6.25 | 6.25 | 6.25 | 6.25 | 152.90 | 3,641.97 | >5,120 |

Boost
Day

| ID# | Priming Immunization + Boost | 66 | 70 | 73 | 80 | 91 | 105 |
|---|---|---|---|---|---|---|---|
| 01R 0044 | pMSINH-FdU + Aerosol MV | 62.03 | 223.67 | 7,794.68 | 17,633.53 | 15,209.88 | 7,419.82 |
| 02R 0158 | pMSINH-FdU + Aerosol MV | >2,560 | >2,560 | >2,560 | >2,560 | >2,560 | >2,560 |
| 01R 2342 | pMSINH-FdU + Aerosol MV | >2,560 | >2,560 | 237.65 | 292.31 | 671.13 | 229.60 |
| 02R 0002 | pMSINH-FdU + IVX908-MV | >2,560 | >2,560 | >2,560 | >2,560 | 2,338.49 | 747.53 |
| 7Q | pMSINH-FdU ++ IVX908-MV | 605.14 | 2,259.18 | 1,036.25 | 335.57 | 268.82 | 155.83 |
| 42Q | pMSINH-FdU ++ IVX908-MV | 800.00 | 1,532.47 | 1,836.76 | — | 800.00 | 700.71 |
| 16Q | CVD1208(pMSIN/HF) + Aerosol MV | 6.25 | 6.25 | 6.25 | 6.25 | 6.25 | 6.25 |
| 11Q | CVD1208(pMSIN/HF) + Aerosol MV | 6.25 | 6.25 | 6.25 | 70.60 | 155.13 | 45.52 |
| 01R 2488 | CVD1208(pMSIN/HF) + Aerosol MV | 6.25 | 6.25 | 6.25 | >80 | >80 | >80 |
| 5Q | PBS + IVX908-MV | 6.25 | 6.25 | 6.25 | 6.25 | 6.25 | 6.25 |
| 25P | PBS + IVX908-MV | 6.25 | 6.25 | 6.25 | 6.25 | 6.25 | 6.25 |
| 01R 0894 | PBS + Aerosol MV | 6.25 | 6.25 | 6.25 | 45.87 | 237.65 | 400.00 |
| 01R 0650 | PBS + Aerosol MV | 6.25 | 6.25 | 6.25 | 17.65 | 15.73 | 6.25 |
| 29Q | pMSINH + Aerosol MV | >5,120 | >5,120 | >5,120 | >5,120 | >5,120 | >5,120 |
| 01R 0072 | pMSINH + Aerosol MV | >5,120 | >5,120 | >5,120 | >5,120 | >5,120 | >5,120 |
| 01R 0742 | pMSINH + Aerosol MV | >5,120 | >5,120 | >5,120 | >5,120 | >5,120 | >5,120 |
| 24Q | pMSINH + IVX908-MV | 3,935.94 | 8,831.26 | 9,036.74 | 4,925.38 | 5,314.84 | 4,074.10 |
| 10Q | pMSINH + IVX908-MV | 6,400.00 | 9,036.74 | 6,073.08 | 5,298.28 | 2 150.54 | 2,926.34 |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An immunogenic composition, comprising an adjuvant and one or more measles virus antigens, wherein the adjuvant comprises a Proteosome and liposaccharide, and wherein at least one measles virus antigen is an H protein.

2. The immunogenic composition according to claim 1 comprising the adjuvant and two or more measles virus antigens comprising an F protein and an H protein.

3. The immunogenic composition according to claim 1 wherein one or more measles virus antigens are recombinant measles antigens.

4. The immunogenic composition according to claim 1, wherein one or more measles antigens is a measles split antigen.

5. The immunogenic composition according to claim 4 wherein the measles split antigen is from a Moraten strain, Schwarz strain, Zagreb strain, or Edmonston strain.

6. The immunogenic composition according to claim 1 wherein the liposaccharide final content by weight as a percentage of Proteosome protein ranges from about 10% to 500%.

7. The immunogenic composition according to claim 1 wherein the Proteosome and liposaccharide are obtained from the same bacteria.

8. The immunogenic composition according to claim 1 wherein the Proteosome and liposaccharide are obtained from different bacteria.

9. The immunogenic composition according to claim 1 wherein the Proteosome is obtained from Neisseria species.

10. The immunogenic composition according to claim 1 wherein the liposaccharide is from Shigella, Plesiomonas, Escherichia, or Salmonella species.

11. The immunogenic composition according to claim 1 wherein the immunogenic composition further comprises one or more additional microbial antigens.

12. The immunogenic composition according to claim 11 wherein the one or more additional microbial antigens is a viral antigen, a bacterial antigen, a parasitic antigen, or a combination thereof.

13. The immunogenic composition according to claim 1 wherein the ratio of Proteosome to measles virus antigen is at least 4:1.

14. The immunogenic composition according to claim 1 wherein the ratio of Proteosome to measles virus antigen is at least 2:1.

15. An immunogenic composition comprising an adjuvant and one or more measles virus antigens, wherein the adjuvant comprises a Proteosome and the at least one measles virus antigen is an H protein.

16. The immunogenic composition according to claim 15 comprising the adjuvant and two or more measles virus antigens comprising an F protein and an H protein.

17. The immunogenic composition according to claim 15 wherein one or more measles virus antigens are recombinant measles antigens.

18. The immunogenic composition according to claim 15 wherein one or more measles virus antigens is a measles split antigen.

19. The immunogenic composition according to claim 18 wherein the measles split antigen is from a Moraten strain, Schwarz strain, Zagreb strain, or Edmonston strain.

20. The immunogenic composition according to claim 15 wherein the Proteosome is from Neisseria meningitidis.

21. The immunogenic composition according to claim 15 wherein the ratio of Proteosome to measles virus antigen is at least 4:1.

22. The immunogenic composition according to claim 15 wherein the ratio of Proteosome to measles virus antigen is at least 2:1.

23. The immunogenic composition according to claim 15 wherein the immunogenic composition further comprises at least one additional microbial antigen.

24. The immunogenic composition according to claim 23 wherein the at least one additional microbial antigen is viral, bacterial, parasitic, or a combination thereof.

25. The immunogenic composition according to claim 21 wherein the Proteosome is obtained from Neisseria meningitidis.

26. The immunogenic composition according to claim 1 wherein the Proteosome is obtained from Neisseria meningitidis, and the liposaccharide is obtained from Shigella flexneri.

27. The immunogenic composition according to either claim 1 or claim 15, further comprising a pharmaceutically acceptable carrier, excipient, or diluent.

28. A method of treating or preventing a measles infection, comprising administering to a subject in need thereof the immunogenic composition according to either claim 1 or claim 15.

29. The method according to claim 28 wherein the immunogenic composition is administered by a route selected from the group consisting of mucosal, enteral, parenteral, transdermal, transmucosal, intranasal, and inhalation.

30. The method according to claim 28 wherein the immunogenic composition is administered intranasally.

31. A method of eliciting an immune response, comprising administering to a subject in need thereof the immunogenic composition according to either claim 1 or claim 15.

32. The method according to claim 31 wherein the immunogenic composition is administered parenterally or intranasally.

33. The method of claim 31 wherein the immune response comprises a mucosal immune response.

34. The method of claim 33 wherein the mucosal immune response comprises production of an IgA immunoglobulin.

35. The method of claim 31 wherein the immune response comprises a cell-mediated response.

36. The method of claim 31 wherein the immune response comprises a systemic humoral response.

37. A method for eliciting an immune response comprising (a) administering to a subject in need thereof a recombinant expression vector comprising at least one promoter operatively linked to a polynucleotide encoding at least one measles virus antigen, followed by (b) administering at least once the immunogenic composition of either claim 1 or claim 15.

38. The method according to claim 37 wherein in step (b) the immunogenic composition of claim 1 is administered.

39. The method of claim 37 wherein the immunogenic composition is administered parenterally or intranasally.

40. The method of claim 37 wherein the immune response comprises a mucosal immune response.

41. The method of claim 40 wherein the mucosal immune response comprises production of an IgA immunoglobulin.

42. The method of claim 37 wherein the immune response comprises a cell-mediated response.

43. The method of claim 37 wherein the immune response comprises a systemic humoral response.

44. A method for treating or preventing a measles infection, comprising administering to a subject in need thereof a recombinant expression vector comprising at least one promoter operatively linked to a polynucleotide encoding at least one measles virus antigen, followed by (b) administering at least one time the immunogenic composition of either claim 1 or claim 15.

45. (canceled)

46. The method of claim 44 comprising the composition is administered by a route selected from the group consisting of mucosal, enteral, parenteral, transdermal, transmucosal, intranasal, and inhalation.

47. The method according to claim 44 wherein the immunogenic composition is administered intranasally.
